SEQUENCE LISTING (190 sequences)

SEQ ID NO: 1 - protein, 14 aa, Artificial Sequence
  Synthetic Polypeptide
GSSTGAVTSGNYPN

SEQ ID NO: 2 - protein, 7 aa, Artificial Sequence
  Synthetic Polypeptide
GTKFLAP

SEQ ID NO: 3 - protein, 9 aa, Artificial Sequence
  Synthetic Polypeptide
VLWYSNRWV

SEQ ID NO: 4 - protein, 5 aa, Artificial Sequence
  Synthetic Polypeptide
KYAMN

SEQ ID NO: 5 - protein, 19 aa, Artificial Sequence
  Synthetic Polypeptide
RIRSKYNNYATYYADSVKD

SEQ ID NO: 6 - protein, 14 aa, Artificial Sequence
  Synthetic Polypeptide
HGNFGNSYISYWAY

SEQ ID NO: 7 - protein, 125 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 8 - protein, 109 aa, Artificial Sequence
  Synthetic Polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 9 - protein, 249 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 10 - protein, 17 aa, Artificial Sequence
  Synthetic Polypeptide
KSSQSVLDSSTNKNSLA

SEQ ID NO: 11 - protein, 7 aa, Artificial Sequence
  Synthetic Polypeptide
WASTRES

SEQ ID NO: 12 - protein, 9 aa, Artificial Sequence
  Synthetic Polypeptide
QQSAHFPIT

SEQ ID NO: 13 - protein, 5 aa, Artificial Sequence
  Synthetic Polypeptide
NYGMN

SEQ ID NO: 14 - protein, 17 aa, Artificial Sequence
  Synthetic Polypeptide
WINTYTGEPTYADKFQG

SEQ ID NO: 15 - protein, 13 aa, Artificial Sequence
  Synthetic Polypeptide
WSWSDGYYVYFDY

SEQ ID NO: 16 - protein, 122 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 17 - protein, 122 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 18 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 19 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 20 - protein, 505 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - protein, 530 aa, Artificial Sequence
  Synthetic Polypeptide
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 22 - protein, 993 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 511 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 24 - protein, 16 aa, Artificial Sequence
  Synthetic Polypeptide
RSSQSLVHSDGNTYLS

SEQ ID NO: 25 - protein, 7 aa, Artificial Sequence
  Synthetic Polypeptide
RISRRFS

SEQ ID NO: 26 - protein, 9 aa, Artificial Sequence
  Synthetic Polypeptide
MQSTHVPRT

SEQ ID NO: 27 - protein, 5 aa, Artificial Sequence
  Synthetic Polypeptide
NYGMH

SEQ ID NO: 28 - protein, 17 aa, Artificial Sequence
  Synthetic Polypeptide
VIWYDGSDKYYADSVRG

SEQ ID NO: 29 - protein, 15 aa, Artificial Sequence
  Synthetic Polypeptide
DGYDILTGNPRDFDY

SEQ ID NO: 30 - protein, 124 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 31 - protein, 112 aa, Artificial Sequence
  Synthetic Polypeptide
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 32 - protein, 251 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 33 - protein, 506 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 34 - protein, 5 aa, Artificial Sequence
  Synthetic Polypeptide
DYYMT

SEQ ID NO: 35 - protein, 17 aa, Artificial Sequence
  Synthetic Polypeptide
YISSSGSTIYYADSVKG

SEQ ID NO: 36 - protein, 8 aa, Artificial Sequence
  Synthetic Polypeptide
DRNSHFDY

SEQ ID NO: 37 - protein, 11 aa, Artificial Sequence
  Synthetic Polypeptide
RASQGINTWLA

SEQ ID NO: 38 - protein, 7 aa, Artificial Sequence
  Synthetic Polypeptide
GASGLQS

SEQ ID NO: 39 - protein, 9 aa, Artificial Sequence
  Synthetic Polypeptide
QQAKSFPRT

SEQ ID NO: 40 - protein, 117 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 41 - protein, 107 aa, Artificial Sequence
  Synthetic Polypeptide
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 42 - protein, 239 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 43 - protein, 494 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 982 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 982 aa, Artificial Sequence; identical to SEQ ID NO: 44
  Synthetic Polypeptide

SEQ ID NO: 46 - protein, 5 aa, Artificial Sequence
  Synthetic Polypeptide
SYGMH

SEQ ID NO: 47 - protein, 17 aa, Artificial Sequence
  Synthetic Polypeptide
FIWYEGSNKYYAESVKD

SEQ ID NO: 48 - protein, 16 aa, Artificial Sequence
  Synthetic Polypeptide
RAGIIGTIGYYYGMDV

SEQ ID NO: 49 - protein, 11 aa, Artificial Sequence
  Synthetic Polypeptide
SGDRLGEKYTS

SEQ ID NO: 50 - protein, 7 aa, Artificial Sequence
  Synthetic Polypeptide
QDTKRPS

SEQ ID NO: 51 - protein, 9 aa, Artificial Sequence
  Synthetic Polypeptide
QAWESSTVV

SEQ ID NO: 52 - protein, 125 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 53 - protein, 107 aa, Artificial Sequence
  Synthetic Polypeptide
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 54 - protein, 247 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 55 - protein, 507 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 56 - protein, 7 aa, Artificial Sequence
  Synthetic Polypeptide
NARMGVS

SEQ ID NO: 57 - protein, 16 aa, Artificial Sequence
  Synthetic Polypeptide
HIFSNDEKSYSTSLKN

SEQ ID NO: 58 - protein, 14 aa, Artificial Sequence
  Synthetic Polypeptide
IVGYGSGWYGFFDY

SEQ ID NO: 59 - protein, 11 aa, Artificial Sequence
  Synthetic Polypeptide
RASQGIRNDLG

SEQ ID NO: 60 - protein, 7 aa, Artificial Sequence
  Synthetic Polypeptide
AASTLQS

SEQ ID NO: 61 - protein, 9 aa, Artificial Sequence
  Synthetic Polypeptide
LQHNSYPLT

SEQ ID NO: 62 - protein, 124 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 63 - protein, 107 aa, Artificial Sequence
  Synthetic Polypeptide
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 64 - protein, 246 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 65 - protein, 501 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 66 - protein, 989 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 5 aa, Artificial Sequence
  Synthetic Polypeptide
SYYWS

SEQ ID NO: 68 - protein, 16 aa, Artificial Sequence
  Synthetic Polypeptide
YVYYSGTTNYNPSLKS

SEQ ID NO: 69 - protein, 10 aa, Artificial Sequence
  Synthetic Polypeptide
IAVTGFYFDY

SEQ ID NO: 70 - protein, 12 aa, Artificial Sequence
  Synthetic Polypeptide
RASQRVNNNYLA

SEQ ID NO: 71 - protein, 7 aa, Artificial Sequence
  Synthetic Polypeptide
GASSRAT

SEQ ID NO: 72 - protein, 9 aa, Artificial Sequence
  Synthetic Polypeptide
QQYDRSPLT

SEQ ID NO: 73 - protein, 118 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 74 - protein, 108 aa, Artificial Sequence
  Synthetic Polypeptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 75 - protein, 241 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 76 - protein, 496 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 77 - protein, 982 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 5 aa, Artificial Sequence
  Synthetic Polypeptide
SYGMH

SEQ ID NO: 79 - protein, 17 aa, Artificial Sequence
  Synthetic Polypeptide
VISYEGSNKYYAESVKG

SEQ ID NO: 80 - protein, 13 aa, Artificial Sequence
  Synthetic Polypeptide
DRGTIFGNYGLEV

SEQ ID NO: 81 - protein, 16 aa, Artificial Sequence
  Synthetic Polypeptide
RSSQSLLHKNAFNYLD

SEQ ID NO: 82 - protein, 7 aa, Artificial Sequence
  Synthetic Polypeptide
LGSNRAS

SEQ ID NO: 83 - protein, 9 aa, Artificial Sequence
  Synthetic Polypeptide
MQALQTPFT

SEQ ID NO: 84 - protein, 122 aa, Artificial Sequence
  Synthetic Polypeptide
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 85 - protein, 112 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 86 - protein, 503 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 87 - protein, 991 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 5 aa, Artificial Sequence
  Synthetic Polypeptide
NHIIH

SEQ ID NO: 89 - protein, 17 aa, Artificial Sequence
  Synthetic Polypeptide
YINPYPGYHAYNEKFQG

SEQ ID NO: 90 - protein, 12 aa, Artificial Sequence
  Synthetic Polypeptide
DGYYRDTDVLDY

SEQ ID NO: 91 - protein, 11 aa, Artificial Sequence
  Synthetic Polypeptide
QASQDISNYLN

SEQ ID NO: 92 - protein, 7 aa, Artificial Sequence
  Synthetic Polypeptide
YTSRLHT

SEQ ID NO: 93 - protein, 9 aa, Artificial Sequence
  Synthetic Polypeptide
QQGNTLPWT

SEQ ID NO: 94 - protein, 121 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 95 - protein, 107 aa, Artificial Sequence
  Synthetic Polypeptide
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 96 - protein, 243 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 97 - protein, 986 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 504 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 99 - protein, 986 aa, Artificial Sequence; identical to SEQ ID NO: 97
  Synthetic Polypeptide

SEQ ID NO: 100 - protein, 5 aa, Artificial Sequence
  Synthetic Polypeptide
DYYMY

SEQ ID NO: 101 - protein, 17 aa, Artificial Sequence
  Synthetic Polypeptide
IISDAGYYTYYSDIIKG

SEQ ID NO: 102 - protein, 12 aa, Artificial Sequence
  Synthetic Polypeptide
GFPLLRHGAMDY

SEQ ID NO: 103 - protein, 11 aa, Artificial Sequence
  Synthetic Polypeptide
KASQNVDANVA

SEQ ID NO: 104 - protein, 7 aa, Artificial Sequence
  Synthetic Polypeptide
SASYVYW

SEQ ID NO: 105 - protein, 9 aa, Artificial Sequence
  Synthetic Polypeptide
QQYDQQLIT

SEQ ID NO: 106 - protein, 121 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 107 - protein, 107 aa, Artificial Sequence
  Synthetic Polypeptide
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 108 - protein, 243 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 109 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 986 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 984 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 5 aa, Artificial Sequence
  Synthetic Polypeptide
DYYMY

SEQ ID NO: 113 - protein, 17 aa, Artificial Sequence
  Synthetic Polypeptide
IISDGGYYTYYSDIIKG

SEQ ID NO: 114 - protein, 12 aa, Artificial Sequence
  Synthetic Polypeptide
GFPLLRHGAMDY

SEQ ID NO: 115 - protein, 11 aa, Artificial Sequence
  Synthetic Polypeptide
KASQNVDTNVA

SEQ ID NO: 116 - protein, 7 aa, Artificial Sequence
  Synthetic Polypeptide
SASYVYW

SEQ ID NO: 117 - protein, 9 aa, Artificial Sequence
  Synthetic Polypeptide
QQYDQQLIT

SEQ ID NO: 118 - protein, 121 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 119 - protein, 107 aa, Artificial Sequence
  Synthetic Polypeptide
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 120 - protein, 243 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 121 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 122 - protein, 986 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 5 aa, Artificial Sequence
  Synthetic Polypeptide
TYAMS

SEQ ID NO: 124 - protein, 17 aa, Artificial Sequence
  Synthetic Polypeptide
AISGSGGRTFYAESVEG

SEQ ID NO: 125 - protein, 11 aa, Artificial Sequence
  Synthetic Polypeptide
HDYSNYPYFDY

SEQ ID NO: 126 - protein, 12 aa, Artificial Sequence
  Synthetic Polypeptide
RASQSVRSTYLA

SEQ ID NO: 127 - protein, 7 aa, Artificial Sequence
  Synthetic Polypeptide
GASSRAT

SEQ ID NO: 128 - protein, 9 aa, Artificial Sequence
  Synthetic Polypeptide
QQYGDLPFT

SEQ ID NO: 129 - protein, 120 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 130 - protein, 108 aa, Artificial Sequence
  Synthetic Polypeptide
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 131 - protein, 986 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 5 aa, Artificial Sequence
  Synthetic Polypeptide
GYYMH

SEQ ID NO: 133 - protein, 17 aa, Artificial Sequence
  Synthetic Polypeptide
WINPNSGGTKYAQKFQG

SEQ ID NO: 134 - protein, 16 aa, Artificial Sequence
  Synthetic Polypeptide
DRITVAGTYYYYGMDV

SEQ ID NO: 135 - protein, 11 aa, Artificial Sequence
  Synthetic Polypeptide
RASQGVNNWLA

SEQ ID NO: 136 - protein, 7 aa, Artificial Sequence
  Synthetic Polypeptide
TASSLQS

SEQ ID NO: 137 - protein, 9 aa, Artificial Sequence
  Synthetic Polypeptide
QQANSFPIT

SEQ ID NO: 138 - protein, 125 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 139 - protein, 107 aa, Artificial Sequence
  Synthetic Polypeptide
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 140 - protein, 247 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 141 - protein, 502 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 142 - protein, 990 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 125 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 144 - protein, 107 aa, Artificial Sequence; identical to SEQ ID NO: 139
  Synthetic Polypeptide

SEQ ID NO: 145 - protein, 247 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 146 - protein, 502 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 990 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 5 aa, Artificial Sequence
  Synthetic Polypeptide
NHGMH

SEQ ID NO: 149 - protein, 17 aa, Artificial Sequence
  Synthetic Polypeptide
GIWSEGSNKYYADAVKG

SEQ ID NO: 150 - protein, 12 aa, Artificial Sequence
  Synthetic Polypeptide
ATYTTGWSYFDY

SEQ ID NO: 151 - protein, 11 aa, Artificial Sequence
  Synthetic Polypeptide
SGDKLGDKYAS

SEQ ID NO: 152 - protein, 7 aa, Artificial Sequence
  Synthetic Polypeptide
QDAKRPS

SEQ ID NO: 153 - protein, 9 aa, Artificial Sequence
  Synthetic Polypeptide
QAFHQSTWV

SEQ ID NO: 154 - protein, 121 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSS

SEQ ID NO: 155 - protein, 106 aa, Artificial Sequence
  Synthetic Polypeptide
SYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVL

SEQ ID NO: 156 - protein, 985 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 5 aa, Artificial Sequence
  Synthetic Polypeptide
NHAMH

SEQ ID NO: 158 - protein, 17 aa, Artificial Sequence
  Synthetic Polypeptide
GIWSEGSNKYYAESVKG

SEQ ID NO: 159 - protein, 12 aa, Artificial Sequence
  Synthetic Polypeptide
ATYTTGWSYFDY

SEQ ID NO: 160 - protein, 11 aa, Artificial Sequence
  Synthetic Polypeptide
SGDKLGDKYAS

SEQ ID NO: 161 - protein, 7 aa, Artificial Sequence
  Synthetic Polypeptide
QDRKRPS

SEQ ID NO: 162 - protein, 9 aa, Artificial Sequence
  Synthetic Polypeptide
QAYDASTWV

SEQ ID NO: 163 - protein, 121 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSS

SEQ ID NO: 164 - protein, 106 aa, Artificial Sequence
  Synthetic Polypeptide
SYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVL

SEQ ID NO: 165 - protein, 985 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 5 aa, Artificial Sequence
  Synthetic Polypeptide
GYYWS

SEQ ID NO: 167 - protein, 16 aa, Artificial Sequence
  Synthetic Polypeptide
DIDASGSTKYNPSLKS

SEQ ID NO: 168 - protein, 12 aa, Artificial Sequence
  Synthetic Polypeptide
KKYSTVWSYFDN

SEQ ID NO: 169 - protein, 11 aa, Artificial Sequence
  Synthetic Polypeptide
SGDKLGDKYAS

SEQ ID NO: 170 - protein, 7 aa, Artificial Sequence
  Synthetic Polypeptide
QDRKRPS

SEQ ID NO: 171 - protein, 9 aa, Artificial Sequence
  Synthetic Polypeptide
QAWGSSTAV

SEQ ID NO: 172 - protein, 120 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 173 - protein, 106 aa, Artificial Sequence
  Synthetic Polypeptide
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 174 - protein, 984 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 5 aa, Artificial Sequence
  Synthetic Polypeptide
GYYWS

SEQ ID NO: 176 - protein, 16 aa, Artificial Sequence
  Synthetic Polypeptide
DIDYSGSTKYNPSLKS

SEQ ID NO: 177 - protein, 12 aa, Artificial Sequence
  Synthetic Polypeptide
KKYSTVWSYFDY

SEQ ID NO: 178 - protein, 11 aa, Artificial Sequence
  Synthetic Polypeptide
SGDKLGDKYAN

SEQ ID NO: 179 - protein, 7 aa, Artificial Sequence
  Synthetic Polypeptide
HDNKRPS

SEQ ID NO: 180 - protein, 9 aa, Artificial Sequence
  Synthetic Polypeptide
QAYGISSAV

SEQ ID NO: 181 - protein, 120 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSS

SEQ ID NO: 182 - protein, 106 aa, Artificial Sequence
  Synthetic Polypeptide
SYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVL

SEQ ID NO: 183 - protein, 984 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSSGGGGSGGGGSGGGGSSYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 989 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 987 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 501 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 187 - protein, 989 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 987 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 250 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 190 - protein, 250 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

(19) United States
(12) Patent Application Publication
(10) Pub. No.: US 2023/0167176 A1
(43) Pub. Date: Jun. 1, 2023

(54) PHARMACEUTICAL FORMULATION

(71) Applicant: AMGEN INC., Thousand Oaks, CA (US)

(72) Inventors: Twinkle R. Christian, Thousand Oaks, CA (US); Bharadwaj Jagannathan, Thousand Oaks, CA (US)

(73) Assignee: AMGEN INC., Thousand Oaks, CA (US)

(21) Appl. No.: 17/921,794

(22) PCT Filed: Apr. 28, 2021
(86) PCT No.: PCT/US21/29545

§ 371 Date: Oct. 27, 2022

Related U.S. Application Data
(60) Provisional application No. 63/017,061, filed on Apr. 29, 2020.

(51) Int. Cl. C07K 16/28; C07K 16/30; A61K 47/18; A61K 47/26

(52) U.S. Cl. CPC C07K 16/2809; C07K 16/2803; C07K 16/30; C07K 16/2863; C07K 16/2878; C07K 16/2875; C07K 16/28; A61K 47/183; A61K 47/26; C07K 2317/31; C07K 2317/622

(57) ABSTRACT

Described herein are stable high concentration bispecific antibody construct formulations, wherein the formulations have less than 2% high molecular weight species of the bispecific antibody construct in the formulation under storage conditions at various time points.

### FIELD OF THE INVENTION

The present disclosure is in the field of stable high concentration bispecific antibody construct formulations.

### INCORPORATION BY REFERENCE

Incorporated by reference in its entirety is a computer-readable nucleotide/amino acid sequence listing submitted concurrently herewith and identified as follows: ASCII (text) file named “54911_Seqlisting.txt”, 345,249 bytes created Apr. 28, 2021.

### BACKGROUND

Protein-based pharmaceuticals, such as recombinant proteins, can now be obtained in high purity when first manufactured due to advances in commercial scale purification processes. However, proteins are only marginally stable and are highly susceptible to degradation, both chemical and physical. Chemical degradation refers to modifications involving covalent bonds, such as deamidation, oxidation, cleavage, clipping/fragmentation, formation of new disulfide bridges, hydrolysis, isomerization, or deglycosylation. Physical degradation includes protein unfolding, undesirable adsorption to surfaces, and aggregation. Dealing with these physical and chemical instabilities is one of the most challenging tasks in the development of protein pharmaceuticals (Chi et al., Pharm Res, Vol. 20, No. 9, September 2003, pp. 1325-1336, Roberts, Trends Biotechnol. 2014 July; 32(7):372-80).

Half-life extended antibody constructs such as bispecific T cell engagers (BiTE®) comprising a half-life extending modality such as Fc-molecules have to be protected against protein aggregation and/or other degradation events. Protein aggregation of BiTE® molecules is problematic because it can impair biological activity of the therapeutic proteins. Moreover, aggregation of BiTE® molecules may decrease product yield due to elaborate purification steps that are required to remove the aggregates from the end product. More recently, there has also been growing concern and evidence that the presence of aggregated proteins (even humanized or fully human proteins) can significantly increase the risk that a patient will develop an immune response to the active protein monomer, resulting in the formation of neutralizing antibodies and drug resistance, or other adverse side effects (Mahler J Pharm Sci. 2009 September; 98(9):2909-34).

Aggregation is the main type of physical instability observed in high concentration formulations. It is the assembly from initially native and folded proteins into high molecular weight species (HMW). Currently, protein aggregates are removed as impurities mainly in the polishing steps of downstream processing. However, in cases of high levels of HMW species, removing significant amount of HMW not only results in substantial yield loss but also makes the design of a robust downstream process challenging (Chi et al., Pharm Res, Vol. 20, No. 9, September 2003, pp. 1325-1336).

### SUMMARY

The present disclosure provides stable formulations comprising a high concentration (i.e., greater than 10 mg/mL) of bispecific antibody constructs (e.g., BiTE® molecules), wherein less than approximately 3% of the bispecific antibody construct exists as high molecular weight (HMW) species in the formulation. Formulations comprising high concentration of antibodies, while desirable, are generally difficult to stabilize under storage conditions. It was surprisingly found that a formulation according to the present disclosure comprising a bispecific antibody construct at a higher concertation than expected is stable, without requiring the presence of a preservative or stabilizing agent.

In some embodiments, the formulation is a lyophilized formulation. In some embodiments, 2.5% or less (e.g., 2.5%, or 2.0%, or 1.9%, or 1.8%, or 1.7%, or 1.6%, or 1.5%, or 1.4%, or 1.3%, or 1.2%, or 1.1%, or 1.0%, or 0.5%) of the bispecific antibody construct exists as HMW species in the lyophilized formulation. In some embodiments, the amount of HMW species in the lyophilized formulation increases less than 1%, (e.g., 0.9%, 0.8%, 0.7%, 0.6%, 0.5%, 0.4%, 0.3%, 0.2%, 0.1%) upon storage at 4° C. for one month or more (e.g., for one month, for three months, or for six months). In some embodiments, upon storage at 4° C. for one month or more (e.g., for one month, for three months, or for six months), the amount of HMW species in the formulation increases approximately between 0.1% and 0.4% (e.g., 0.1%, 0.2%, 0.3%, or 0.4%). In some embodiments, the amount of HMW species in the lyophilized formulation increases less than 1%, (e.g., 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, upon storage at 40° C. for one week or more (e.g., for one week, for two weeks, for one month or for three months) the amount of HMW species in the lyophilized formulation increases approximately between 0.1% and 0.7% (e.g., 0.1%, 0.2%, 0.3%, 0.4%, 0.5%, 0.6%, or 0.7%).

In some embodiments, less than 2%, (e.g., 1.9%, 1.8%, 1.7%, 1.6%, 1.5%, 1.4%, 1.3%, 1.2%, 1.1%, 1%, or 0.5%) of the bispecific antibody construct exists as low molecular weight (LMW) species in the lyophilized formulation. In some embodiments, the amount of LMW species in the lyophilized formulation increases less than 2%, (e.g., 1.9%, 1.8%, 1.7%, 1.6%, 1.5%, 1.4%, 1.3%, 1.2%, 1.1%, 1%, or 0.5%) upon storage at 4° C. for one month or more (e.g., for one month, for three months, or for six months). In some embodiments, upon storage at 4° C. for one month or more (e.g., for one month, for three months, or for six months), the amount of LMW species in the formulation increases approximately between 0.1% and 0.7% (e.g., 0.1%, 0.2%, 0.3%, 0.4%, 0.5%, 0.6%, or 0.7%). In some embodiments, the amount of LMW species in the lyophilized formulation increases less than 1%, (e.g., 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, upon storage at 40° C. for one week or more (e.g., for one week, for two weeks, for one month or for three months) the amount of LMW species in the lyophilized formulation increases approximately between 0.1% and 0.7% (e.g., 0.1%, 0.2%, 0.3%, 0.4%, 0.5%, 0.6%, or 0.7%).

In some embodiments, the percent of intact BiTE® molecule (i.e., main peak species) in the lyophilized formulation is greater than 95% of the total protein content in the formulation.

In some embodiments, the lyophilized formulation is stable upon storage at about 4° C. for one month, and the amount of HMW species in the formulation increases approximately between 0.1% to 0.7% (e.g., 0.1%, or 0.2%, or 0.3%, or 0.4%, or 0.5%, or 0.6%, or 0.7%) while in storage for at least one month. In some embodiments, the lyophilized formulation is stable upon storage at about 4° C. for three months, and the amount of HMW species in the formulation increases approximately between 0.0% to 0.2% (e.g., 0%, or 0.1%, or 0.2%), while in storage for at least three months. In some embodiments, the lyophilized formulation is stable upon storage at about 4° C. for six months, and the amount of HMW species in the formulation increases approximately between 0.0% to 0.4% (e.g., 0%, or 0.1%, or 0.2%, or 0.3%, or 0.4%), while in storage for at least six months.

In some embodiments, the lyophilized formulation is stable upon storage at about 4° C. for at least one month, three months and six months, and the percent of intact BiTE® molecule is above 95% of the total protein content during that time period in storage.

In some embodiments, the formulation is a liquid formulation. In some embodiments, less than 3% (e.g., 2.5% or 2%, or 1.5%, or 1%, or 0.5%) of the bispecific antibody construct exists as HMW species in the liquid formulation. In some embodiments, the amount of HMW species in the liquid formulation increases less than 3% (e.g., 3%, 2.5%, 2%, 1%, or 0.5%) upon storage at 4° C. for one month or more (e.g., for one month, for three months, for six months, or for one year). In some embodiments, upon storage at 4° C. for one month or more (e.g., for one month, for three months, for six months or for one year), the amount of HMW species in the formulation increases approximately between 0.1% and 1% (e.g., 0.1%, or 0.2%, or 0.3%, or 0.4%, or 0.5%, or 0.6%, or 0.7%, or 0.8%, or 0.9%, or 1%). In some embodiments, the amount of HMW species in the liquid formulation increases less than 5% (e.g., 4.5%, or 4%, or 3.5%, or 3%, or 2.5%, or 2%, or 1.5%, or 1%, or 0.5%) upon storage at 40° C. for one week or more (e.g., for one week, for two weeks, for one month or for three months). In some embodiments, upon storage at 40° C. for one week or more (e.g., for one week, for two weeks, for one month or for three months) the amount of HMW species in the liquid formulation increases approximately between 0.1% and 5% (e.g., 0.1%, or 0.2%, or 0.3%, or 0.4%, or 0.5%, or 0.6%, or 0.7%, or 0.8%, or 0.9%, or 1%, or 1.5%, or 2%, or 2.5%, or 3%, or 3.5%, or 4%, or 4.5% or 5%).

In some embodiments, less than 2%, (e.g., 1.9%, or 1.8%, or 1.7%, or 1.6%, or 1.5%, or 1.4%, or 1.3%, or 1.2%, or 1.1%, or 1%, or 0.9%, or 0.8%, or 0.7%, or 0.6%, or 0.5%) of the bispecific antibody construct exists as low molecular weight (LMW) species in the liquid formulation. In some embodiments, the amount of LMW species in the liquid formulation increases less than 2%, (e.g., 1.9%, or 1.8%, or 1.7%, or 1.6%, or 1.5%, or 1.4%, or 1.3%, or 1.2%, or 1.1%, or 1%, or 0.5%, 0.4%, or 0.3%, or 0.2% or 0.1%) upon storage at 4° C. for one month or more (e.g., for one month, for three months, for six months, or for twelve months). In some embodiments, upon storage at 4° C. for one month or more (e.g., for one month, for three months, for six months, or for twelve months), the amount of LMW species in the liquid formulation increases approximately between 0.1% and 0.7% (e.g., 0.1%, 0.2%, 0.3%, 0.4%, 0.5%, 0.6%, or 0.7%). In some embodiments, the amount of LMW species in the liquid formulation increases less than 7%, (e.g., 6%, or 5%, or 4%, or 3%, or 2%, or 1%, or 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, upon storage at 40° C. for one week or more (e.g., for one week, for two weeks, for one month or for three months) the amount of LMW species in the lyophilized formulation increases approximately between 0.1% and 7% (e.g., 0.1%, 0.2%, 0.3%, 0.4%, 0.5%, 0.6%, or 0.7%, or 0.8%, or 0.9%, or 1%, or 1.5%, or 2%, or 3%, or 5%, or 6%, or 7%).

In some embodiments, the percent of intact BiTE® molecule (i.e., main peak species) in the liquid formulation is greater than 96% of the total protein content in the formulation.

In some embodiments, the liquid formulation is stable upon storage at about 4° C. for one month, and the amount of HMW species in the formulation increases approximately between 0.1% to 0.4% (e.g., 0.1%, or 0.2%, or 0.3%, or 0.4%). In some embodiments, the liquid formulation is stable upon storage at about 4° C. for three months, and the amount of HMW species in the formulation increases approximately between 0.0% to 0.3% (e.g., 0%, or 0.1%, or 0.2%, or 0.3%), while in storage for at least three months. In some embodiments, the liquid formulation is stable upon storage at about 4° C. for six months, and the amount of HMW species in the formulation increases approximately between 0.0% to 0.6% (e.g., 0%, or 0.1%, or 0.2%, or 0.3%, or 0.4%, or 0.5%, or 0.6%), while in storage for at least six months. In some embodiments, the liquid formulation is stable upon storage at about 4° C. for twelve months, and the amount of HMW species in the formulation increases approximately between 0.0% to 0.2% (e.g., 0%, or 0.1%, or 0.2%), while in storage for at least twelve months.

In some embodiments, the lyophilized formulation is stable upon storage at about 4° C. for one month, three months, six months, and twelve months, and the percent of intact BiTE® molecule is above 96% of the total protein content.

In some embodiments, the antibody binding protein is a bispecific antibody construct comprising a first binding domain that binds to a target cell surface antigen, a second binding domain that binds to human CD3 on the surface of a T cell.

In some embodiments, the bispecific antibody construct further comprises a third domain comprising, in an amino to carboxyl order, hinge-CH2 domain-CH3 domain-linker-hinge-CH2 domain-CH3 domain. In some embodiments, each of the first and second binding domains of the bispecific antibody construct comprises a VH region and a VL region.

In some embodiments, the bispecific antibody construct is a single chain antibody construct.

In some embodiments, the bispecific antibody construct binds to a target cell surface antigen, such as, CDH19, MSLN, DLL3, FLT3, EGFRvlll, BCMA, PSMA, CD33, CD19, CD70, CLDN18.2 or MUC17.

In some embodiments, the first binding domain of the bispecific antibody construct comprises a set of 6 CDRs set forth in (a) SEQ ID NOs: 24-29, (b) SEQ ID NOs: 34-39, (c) SEQ ID NOs: 78-83, (d) SEQ ID NOs: 10-15, (e) SEQ ID NOs: 46-51, (f) SEQ ID NOs: 88-93, (g) SEQ ID NOs: 67-72, (h) SEQ ID NOs: 56-61, (i) SEQ ID NOs: 112-117, (j) SEQ ID NOs: 100-105, (k) SEQ ID NOs:148-153, SEQ ID NOs: 157-162, SEQ ID NOs: 166-171, or SEQ ID NOs: 175-180, (1) SEQ ID NOs:132-137, or (m) SEQ ID NOs: 123-128.

In some embodiments, the first binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 30, 40, 84, 16 or 17, 52, 94, 73, 62, 118, 154,163, 172, 181, 106, 138, 143, or 129. In some embodiments, the first binding domain of the bispecific antibody construct comprises a VH comprising the amino acid sequence set forth in SEQ ID NO: 30, 40, 84, 16 or 17, 52, 94, 73, 62, 118, 154, 163, 172, 181, 106, 138, 143, or 129.

In some embodiments, the first binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 31, 41, 85, 18, 19, 53, 95, 74, 63, 119, 155, 164, 173, 182, 107, 139, 144, or 130. In some embodiments, the first binding domain of the bispecific antibody construct comprises a VL comprising the amino acid sequence set forth in SEQ ID NO: 31, 41, 85, 18, 19, 53, 95, 74, 63, 119, 155, 164, 173, 182, 107, 139, 144, or 130.

In some embodiments, wherein the first binding domain comprises (a) a VH region comprising an amino acid sequence set forth in SEQ ID NO: 30 and a VL region comprising an amino acid sequence set forth in SEQ ID NO: 31; (b) a VH region comprising an amino acid sequence set forth in SEQ ID NO: 40 and a VL region comprising an amino acid sequence set forth in SEQ ID NO: 41; (c) a VH region comprising an amino acid sequence set forth in SEQ ID NO: 84 and a VL region comprising an amino acid sequence set forth in SEQ ID NO: 85; (d) a VH region comprising an amino acid sequence set forth in SEQ ID NO: 16 or 17 and a VL region comprising an amino acid sequence set forth in SEQ ID NO: 18 or 19; (e) a VH region comprising an amino acid sequence set forth in SEQ ID NO: 52 and a VL region comprising an amino acid sequence set forth in SEQ ID NO: 53; (f) a VH region comprising an amino acid sequence set forth in SEQ ID NO: 94 and a VL region comprising an amino acid sequence set forth in SEQ ID NO: 95; (g) a VH region comprising an amino acid sequence set forth in SEQ ID NO: 73 and a VL region comprising an amino acid sequence set forth in SEQ ID NO: 74; (h) a VH region comprising an amino acid sequence set forth in SEQ ID NO: 62 and a VL region comprising an amino acid sequence set forth in SEQ ID NO: 63; (i) a VH region comprising an amino acid sequence set forth in SEQ ID NO: 118 and a VL region comprising an amino acid sequence set forth in SEQ ID NO: 119; (j) a VH region comprising an amino acid sequence set forth in SEQ ID NO: 154, 163, 172 or 181, and a VL region comprising an amino acid sequence set forth in SEQ ID NO: 155, 164, 173 or 182; (k) a VH region comprising an amino acid sequence set forth in SEQ ID NO: 106 and a VL region comprising an amino acid sequence set forth in SEQ ID NO: 107; (1) a VH region comprising an amino acid sequence set forth in SEQ ID NO: 138 or 143, and a VL region comprising an amino acid sequence set forth in SEQ ID NO: 139 or 144; or (m) a VH region comprising an amino acid sequence set forth in SEQ ID NO: 129 and a VL region comprising an amino acid sequence set forth in SEQ ID NO: 130.

In some embodiments, the second binding domain of the bispecific antibody construct comprises a set of 6 CDRs set forth in SEQ ID NOs: 1-6.

In some embodiments, the second binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 7. In some embodiments, the second binding domain of the bispecific antibody construct comprises a VH comprising the amino acid sequence set forth in SEQ ID NO: 7.

In some embodiments, the second binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 8. In some embodiments, the second binding domain of the bispecific antibody construct comprises a VL comprising the amino acid sequence set forth in SEQ ID NO: 8.

In some embodiments, wherein the second binding domain comprises (a) a VH region comprising an amino acid sequence set forth in SEQ ID NO: 7 and a VL region comprising an amino acid sequence set forth in SEQ ID NO: 8.

In some embodiments, the bispecific antibody construct comprises a first binding domain that binds CD19 comprising an anti-CD19 variable light domain comprising the amino acid sequence of SEQ ID NO: 85 and an anti-CD19 variable heavy domain comprising the amino acid sequence of SEQ ID NO: 84, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 86 a second binding domain comprising the amino acid sequence of SEQ ID NO: 9. In some embodiments, the bispecific antibody construct comprises the amino acid sequence set forth in SEQ ID NO: 87.

In some embodiments, the bispecific antibody construct comprises a first binding domain that binds MSLN comprising an anti-MSLN variable light domain comprising the amino acid sequence of SEQ ID NO: 41 and an anti-MSLN variable heavy domain comprising the amino acid sequence of SEQ ID NO: 40, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 42, and a second binding domain comprising the amino acid sequence of SEQ ID NO: 9. In some embodiments, the bispecific antibody construct comprises an amino acid sequence set forth in SEQ ID NO: 43, 44 or 45.

In some embodiments, the bispecific antibody construct comprises a first binding domain that binds DLL3 comprising an anti-DLL3 variable light domain comprising the amino acid sequence of SEQ ID NO: 74 and an anti-DLL3 variable heavy domain comprising the amino acid sequence of SEQ ID NO: 73, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 75, and a second binding domain comprising the amino acid sequence of SEQ ID NO: 9. In some embodiments, the bispecific antibody construct comprises an amino acid sequence set forth in SEQ ID NO: 76 or 77.

In some embodiments, the bispecific antibody construct comprises a first binding domain that binds FLT3 comprising an anti-FLT3 variable light domain comprising the amino acid sequence of SEQ ID NO: 63 and an anti-FLT3 variable heavy domain comprising the amino acid sequence of SEQ ID NO: 62, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 64, a second binding domain comprising the amino acid sequence of SEQ ID NO: 9. In some embodiments, the bispecific antibody construct comprises an amino acid sequence set forth in SEQ ID NO: 65 or 66.

In some embodiments, the bispecific antibody construct comprises a first binding domain that binds EGFRvIII comprising an anti-EGFRvIII variable light domain comprising the amino acid sequence of SEQ ID NO: 31 and an anti-EGFRvIII variable heavy domain comprising the amino acid sequence of SEQ ID NO: 30, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 32, a second binding domain comprising the amino acid sequence of SEQ ID NO: 9. In some embodiments, the bispecific antibody construct comprises an amino acid sequence set forth in SEQ ID NO: 33.

In some embodiments, the bispecific antibody construct comprises a first binding domain that binds BCMA comprising an anti-BCMA variable light domain comprising the amino acid sequence of SEQ ID NO: 95 and an anti-BCMA variable heavy domain comprising the amino acid sequence of SEQ ID NO: 94, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 96, a second binding domain comprising the amino acid sequence of SEQ ID NO: 9. In some embodiments, the bispecific antibody construct comprises an amino acid sequence set forth in SEQ ID NO: 98 or SEQ ID NO: 97.

In some embodiments, the bispecific antibody construct comprises a first binding domain that binds PSMA comprising an anti-PSMA variable light domain comprising the amino acid sequence of SEQ ID NO: 119 or 107 and an anti-PSMA variable heavy domain comprising the amino acid sequence of SEQ ID NO: 118 or 106, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 120 or 108, a second binding domain comprising the amino acid sequence of SEQ ID NO: 9. In some embodiments, the bispecific antibody construct comprises an amino acid sequence set forth in SEQ ID NO: 121, 122, 109, 110 or 111.

In some embodiments, the bispecific antibody construct comprises a first binding domain that binds CD33 comprising an anti-CD33 variable light domain comprising the amino acid sequence of SEQ ID NO: 18 or 19 and an anti-CD33 variable heavy domain comprising the amino acid sequence of SEQ ID NO: 16 or 17, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 189 or 190, a second binding domain comprising the amino acid sequence of SEQ ID NO: 9. In some embodiments, the bispecific antibody construct comprises the amino acid sequence set forth in SEQ ID NO: 20, 21, 22 or 23.

In some embodiments, the bispecific antibody construct comprises a first binding domain that binds CDH19 comprising an anti-CDH19 variable light domain comprising the amino acid sequence of SEQ ID NO: 53 and an anti-CDH19 variable heavy domain comprising the amino acid sequence of SEQ ID NO: 52, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 54, a second binding domain comprising the amino acid sequence of SEQ ID NO: 9. In some embodiments, the bispecific antibody construct comprises the amino acid sequence set forth in SEQ ID NO: 55.

In some embodiments, the bispecific antibody construct comprises a first binding domain that binds MUC17 comprising an anti-MUC17 variable light domain comprising the amino acid sequence of SEQ ID NO: 155, 164, 173, or 182 and an anti-MUC17 variable heavy domain comprising the amino acid sequence of SEQ ID NO: 154, 163, 172, or 181 a second binding domain comprising an anti-CD3 variable heavy domain comprising the amino acid sequence of SEQ ID NO: 7, and an anti-CD3 variable light domain comprising the amino acid sequence of SEQ ID NO: 8. In some embodiments, the bispecific antibody construct comprises the amino acid sequence set forth in SEQ ID NO: 156, 165, 174 or 183.

In some embodiments, the bispecific antibody construct comprises a first binding domain that binds cldn18.2 comprising an anti-cldn18.2 variable light domain comprising the amino acid sequence of SEQ ID NO: 139 or 144 and an anti-cldn18.2 variable heavy domain comprising the amino acid sequence of SEQ ID NO: 138 or 143, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 140 or 145, and a second binding domain comprising the amino acid sequence of SEQ ID NO: 9. In some embodiments, the bispecific antibody construct comprises the amino acid sequence set forth in SEQ ID NO: 141, 142, 146 or 147.

In some embodiments, the bispecific antibody construct comprises a first binding domain that binds CD70 comprising an anti-CD70 variable light domain comprising the amino acid sequence of SEQ ID NO: 130 and an anti-CD70 variable heavy domain comprising the amino acid sequence of SEQ ID NO: 129, a second binding domain comprising an anti-CD3 variable heavy domain comprising the amino acid sequence of SEQ ID NO: 7, and an anti-CD3 variable light domain comprising the amino acid sequence of SEQ ID NO: 8. In some embodiments, the bispecific antibody construct comprises an amino acid sequence set forth in SEQ ID NO: 131.

The pharmaceutical formulation of the disclosure comprises a buffer. In some embodiments, the buffer is acetate, glutamate, citrate, succinate, tartrate, fumarate, maleate, histidine, phosphate, 2-(N-morpholino)ethanesulfonate or a combination thereof. In some embodiments, the buffer is present in the formulation at a concentration ranging from about 5 mM to about 200 mM (or about 10 mM to about 50 mM).

The pharmaceutical formulation of the disclosure comprises a saccharide. In some embodiments, the saccharide is monosaccharide or a disaccharide. In some embodiments, the saccharide is a sugar alcohol (e.g., sorbitol). In some embodiments, the saccharide is sucrose, trehalose, mannitol, sorbitol or a combination thereof. In some embodiments, the saccharide is present in the formulation at a concentration ranging from about 1 to about 15% (w/V) (or about 9 to about 12% (w/V) or about 5% to about 12% (w/V) or about 7% to about 12% (w/V)).

The pharmaceutical formulation of the disclosure comprises a surfactant. In some embodiments, the surfactant is polysorbate 20, polysorbate 40, polysorbate 60, polysorbate 80, poloxamer 188, poloxamer 407, triton X-100, polyoxyethylene, PEG 3350, PEG 4000, or a combination thereof. In some embodiments, the surfactant is present in the formulation at a concentration ranging from 0.004 to about 0.5% (w/V) (or about 0.001 to about 0.01% (w/V), or about 0.001 to about 0.5% (w/V) or about 0.004 to about 0.01% (w/V)).

In some embodiments, the formulation has an osmolarity in the range of about 150 to about 500 mOsm. In some embodiments, the formulation has an osmolarity of no greater than 500 mOsm/L, 450 mOsm/L, 400 mOsm/L, or 350 mOsm/L. In some embodiments, the formulation is close to isotonic, e.g. 250-350 mOsm/L.

The pharmaceutical formulation, in some embodiments, comprises 10 mM glutamate, 9% (w/V) sucrose and 0.01% (w/V) polysorbate 80, and wherein the pH of the pharmaceutical formulation is 4.2. In some embodiments, the bispecific antibody construct is present in the formulation at a concentration ranging from about 10 mg/mL to about 100 mg/mL. In some embodiments, the bispecific antibody construct is present in the formulation at a concentration of 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 30 mg/mL, 35 mg/mL, 40 mg/mL, 45 mg/mL, or 50 mg/mL. In some embodiments, the bispecific antibody is present in the formulation in an amount ranging from about 1000 μg to about 200 mg.

In some embodiments, the pharmaceutical formulation of the disclosure is a liquid formulation.

In another aspect, described herein is a method of treating cancer in a subject in need thereof comprising administering a formulation of the disclosure to the subject.

It should be understood that while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” the feature. It is to be noted that the term “a” or “an” refers to one or more, for example, “an immunoglobulin molecule,” is understood to represent one or more immunoglobulin molecules. As such, the terms “a” (or “an”), “one or more,” and “at least one” can be used interchangeably herein.

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5 etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH.

When the term “about” is used, it means the recited number plus or minus 5%, 10%, 15% or more of that recited number. The actual variation intended is determinable from the context.

In any of the ranges described herein, the endpoints of the range are included in the range. However, the description also contemplates the same ranges in which the lower and/or the higher endpoint is excluded. Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

All references cited herein are hereby incorporated by reference in their entireties.

### BRIEF DESCRIPTION OF THE FIGURES

FIG. 1 provides graphs showing the percent high molecular weight (HMW) species of BiTE®-I, BiTE®-C, and BiTE®-G, each at 20 mg/mL in a lyophilized formulation comprising 10 mM glutamate, 9% w/v sucrose, 0.01% w/v Polysorbate 80, pH 4.2, when assessed by size-exclusion ultra high-performance liquid chromatography (SE-UHPLC) under storage conditions at 4° C. t time points 0, one month and three months.

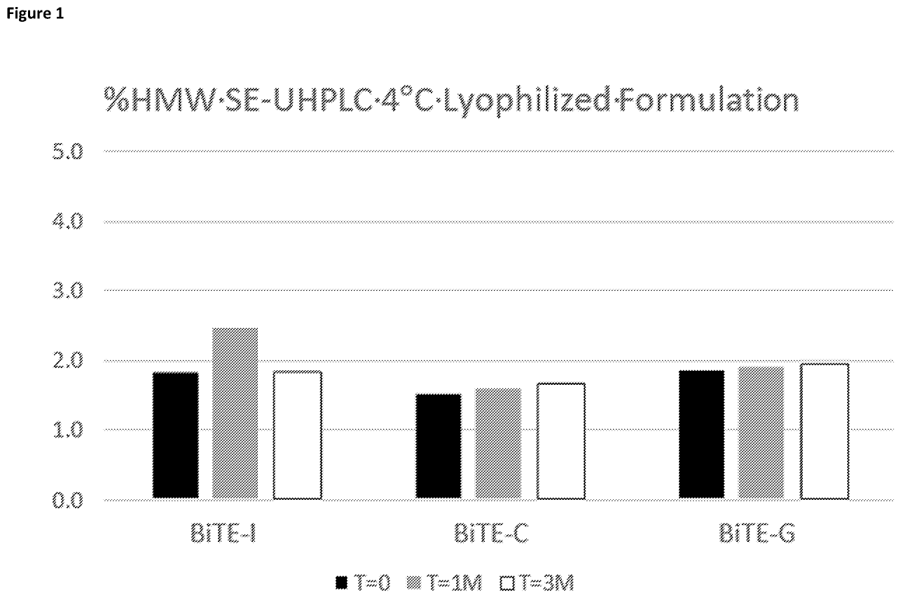

FIG. 2 provides graphs showing the percent high molecular weight (HMW) species of BiTE®-I, BiTE®-C, and BiTE®-G, each at 20 mg/mL in a liquid formulation comprising 10 mM glutamate, 9% w/v sucrose, 0.01% w/v Polysorbate 80, pH 4.2, when assessed by size-exclusion ultra high-performance liquid chromatography (SE-UHPLC) under storage conditions at 4° C. t time points 0, one month and three months.

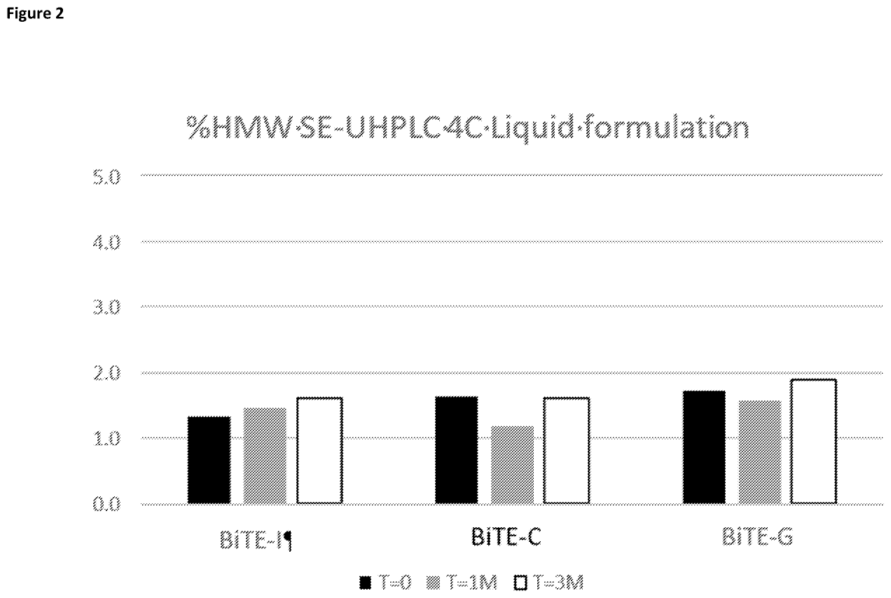

### DETAILED DESCRIPTION

Despite the high quality of current therapeutic biotech products and the resemblance of recombinant human proteins and antibodies to endogenous human proteins, protein instability remains an important concern. There is a critical need in the art to increase stability and reduce aggregation of therapeutic proteins; and optimized pharmaceutical formulations can aid in doing so.

Normally, antibody constructs as described herein are stored and/or employed in a lyophilized formulation at a concentration of about 1 mg/ml (International Publication No. WO 2018/204907) or at most about 8 mg/mL (International Publication No. WO 2018/141910). At higher concentrations, aggregation tendencies are generally observed. The formulations described in International Publication No. WO 2018/204907 having an antibody concentration of 0.5 mg/mL-20 mg/mL include a preservative (such as chlorobutanol or methylparaben, or benzyl alcohol) that attributed to the stabilization of the bispecific antibody construct. In some instances, stability is achieved by other means, such as by lowering the pH of the formulation. For example, International Publication No. WO 2018/141910 discloses that formulations comprising a bispecific antibody construct (5 mg/mL) and having a low pH (i.e., pH 4.0) were more stable than formulations having the same antibody concentration at a more basic pH (e.g., pH 6 or higher). Thus, the low pH attributed to the stability of the formulations. However, as explained herein, formulations (both liquid and lyophilized) comprising high concentration of a bispecific antibody construct (e.g., 20 mg/mL) were unexpectedly stable (e.g., have low percent high molecular weight (HMW) species) at both 4° C. and 40° C. at various time points without requiring the addition of a preservative or other stabilizing agent or having a more acidic pH. The stable high concentration formulations disclosed herein are preferred over the formulations known in the art because it is beneficial to omit non-essential components in pharmaceutical formulations, such as preservatives or other components acting as stabilizing agents.

The present disclosure provides stable formulations comprising a high concentration of bispecific antibody constructs (e.g., BiTE® molecules), wherein less than approximately 2% of the bispecific antibody construct exists as high molecular weight (HMW) species in the formulation.

Within the present disclosure, the term “stability” or “stabilization” relates to the stability of the pharmaceutical formulation in total and in particular to the stability of the active ingredient (e.g. the bispecific antibody construct) itself, specifically during formulation, filling, shipment, storage and administration. A “stable formulation” is one in which the bispecific antibody construct therein essentially retains its physical and/or chemical integrity and biological activity upon storage and during processes (such as freeze/thaw, mechanical mixing and lyophilization). Protein stability can be measured by formation of high molecular weight (HMW) species, loss of enzyme activity, generation of peptide fragments and shift of charge profiles.

The term “aggregation” as used herein refers to the direct mutual attraction between molecules, e.g. via van der Waals forces or chemical bonding. In particular, aggregation is understood as proteins accumulating and clumping together. Aggregates may include amorphous aggregates and oligomers and are typically referred to as high molecular weight (HMW) species, i.e. molecules having a higher molecular weight than product molecules which are non-aggregated molecules.

The term “(protein) aggregate” as used herein generally encompasses protein species of higher molecular weight such as “oligomers” or “multimers” instead of the desired defined species (e.g., a monomer). The term is used interchangeably herein with the terms “high molecular weight” species and “HMW”. Protein aggregates may generally differ in size (ranging from small (dimers) to large assemblies (subvisible or even visible particles) and from the nanometer to micrometer range in diameter), morphology (approximately spherical to fibrillar), protein structure (native vs. non-native/denatured), type of intermolecular bonding (covalent vs. non-covalent), reversibility and solubility. Soluble aggregates cover the size range of roughly 1 to 100 nm, and protein particulates cover subvisible (˜0.1-100 nm) and visible (>100 nm) ranges. All of the aforementioned types protein of aggregates are generally encompassed by the term. The term “(protein) aggregate” thus refers to all kinds physically-associated or chemically linked non-native species of two or more protein monomers.

The term “low molecular weight (LMW)” species as used herein refers to fragments of the bispecific antibody construct.

As used herein, the term “pharmaceutical formulation” relates to a formulation which is suitable for administration to a subject in need thereof. The terms “subject” or “individual” or “animal” or “patient” are used interchangeably herein to refer to any subject, particularly a mammalian subject, for whom administration of the pharmaceutical formulation of the invention is desired. Mammalian subjects include humans, non-human primates, dogs, cats, guinea pigs, rabbits, rats, mice, horses, cattle, cows, and the like, with humans being preferred. The pharmaceutical formulation of the present disclosure is stable and pharmaceutically acceptable, i.e., capable of eliciting the desired therapeutic effect without causing significant undesirable local or systemic effects in the subject to which the pharmaceutical formulation is administered. Pharmaceutically acceptable formulations of the invention may be sterile. Specifically, the term “pharmaceutically acceptable” can mean approved by a regulatory agency or other generally recognized pharmacopoeia for use in animals, and more particularly in humans, but is not limited to those approved by a regulatory agency.

In some embodiments, the disclosure describes formulations comprising a bispecific antibody construct that binds CD3 on human T cells in an amount of at least 10 mg/mL, a buffer, a saccharide, and a surfactant, wherein the formulation has a pH ranging from 4-6. In some embodiments, the bispecific antibody construct co-engages CD3 and one of human CDH19, human MSLN, human DLL3, human FLT3, human EGFRvlll, human BCMA, human PSMA, human CD33, human CD19, human CD70, human CLDN18.2 or human MUC17 in such a manner so as to transiently connect malignant cells with T cells, thereby inducing T cell mediated killing of the bound malignant cell.

Various aspects of the formulations are described below. The use of section headings is merely for the convenience of reading, and not intended to be limiting per se. The entire document is intended to be viewed as a unified disclosure, and it should be understood that all combinations of features described herein are contemplated.

Antigen-Binding Proteins

An “antigen-binding protein” is a protein comprising a domain that binds a specified target antigen (such as CD3 and/or CDH19, MSLN, DLL3, FLT3, EGFRvlll, BCMA, PSMA, CD33, CD19, CD70, CLDN18.2 or MUC17). An antigen-binding protein comprises a scaffold or framework portion that allows the antigen binding domain to adopt a conformation that promotes binding of the antigen-binding protein to the antigen. In exemplary aspects, the antigen-binding protein is an antibody or immunoglobulin, or an antigen-binding antibody fragment.

The term “antibody” refers to an intact antigen-binding immunoglobulin. An “antibody” is a type of an antigen-binding protein. The antibody can be an IgA, IgD, IgE, IgG, or IgM antibody, including any one of IgG1, IgG2, IgG3 or IgG4. In various embodiments, an intact antibody comprises two full-length heavy chains and two full-length light chains. An antibody has a variable region and a constant region. In IgG formats, a variable region is generally about 100-110 or more amino acids, comprises three complementarity determining regions (CDRs), is primarily responsible for antigen recognition, and substantially varies among other antibodies that bind to different antigens. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). The constant region allows the antibody to recruit cells and molecules of the immune system.

In some embodiments, the antibody of the formulation is a bispecific antibody, i.e., an antibody that binds two different targets (e.g., CD3 and a second, different target).

The term “bispecific” as used herein refers to an antibody construct that binds to two different target antigens, i.e., it comprises a first binding domain and a second binding domain, wherein the first binding domain binds to one antigen or target (e.g., the target cell surface antigen), and the second binding domain binds to another antigen or target (e.g. CD3). Accordingly, antibody constructs according to the disclosure comprise specificities for two different antigens or targets. The term “target cell surface antigen” refers to an antigenic structure expressed by a cell and which is present at the cell surface such that it is accessible for an antibody construct as described herein. It may be a protein, preferably the extracellular portion of a protein, or a carbohydrate structure, preferably a carbohydrate structure of a protein, such as a glycoprotein. It is preferably a tumor antigen. The invention also encompasses multispecific antibody constructs such as trispecific antibody constructs, the latter ones including three binding domains, or constructs having more than three (e.g. four, five . . . ) specificities.

Bispecific antibodies and/or antibody constructs as understood herein include, but are not limited to, traditional bispecific immunoglobulins (e.g., BsIgG), IgG comprising an appended antigen-binding domain (e.g., the amino or carboxy termini of light or heavy chains are connected to additional antigen-binding domains, such as single domain antibodies or paired antibody variable domains (e.g., Fv or scFv)), BsAb fragments (e.g., bispecific single chain antibodies), bispecific fusion proteins (e.g., antigen binding domains fused to an effector moiety), and BsAb conjugates. See, e.g., Spiess et al., Molecular Immunology 67(2) Part A: 97-106 (2015), which describes various bispecific formats and is hereby incorporated by reference. Examples of bispecific constructs include, but are not limited to, diabodies, single chain diabodies, tandem scFvs, bispecific T cell engager (BITE®) format (a fusion protein consisting of two single-chain variable fragments (scFvs) joined by a linker), and Fab2 bispecifics, as well as engineered constructs comprising full length antibodies. See, e.g., Chames & Baty, 2009, mAbs 1[6]:1-9; and Holliger & Hudson, 2005, Nature Biotechnology 23[9]:1126-1136; Wu et al., 2007, Nature Biotechnology 25[11]:1290-1297; Michaelson et al., 2009, mAbs 1[2]:128-141; International Patent Publication No. 2009032782 and 2006020258; Zuo et al., 2000, Protein Engineering 13[5]:361-367; U.S. Patent Application Publication No. 20020103345; Shen et al., 2006, J Biol Chem 281[16]:10706-10714; Lu et al., 2005, J Biol Chem 280[20]:19665-19672; and Kontermann, 2012 MAbs 4(2):182, all of which are expressly incorporated herein.

In some embodiments, the formulations described herein comprise a bispecific antibody construct comprises a first binding domain that binds to a target cell surface antigen, a second binding domain that binds to human CD3 on the surface of a T cell, and optionally a third domain comprising, in an amino to carboxyl order, hinge-CH2 domain-CH3 domain-linker-hinge-CH2 domain-CH3 domain. In some embodiments, each of the first and second binding domains comprise a VH region and a VL region.

The term “binding domain” as used herein refers to a domain which (specifically) binds to/interacts with/recognizes a given target epitope or a given target site on the target molecules (antigens), e.g. CDH19, MSLN, DLL3, FLT3, EGFRvlll, BCMA, PSMA, CD33, CD19, CD70, CLDN18.2 or MUC17 and CD3, respectively. The structure and function of the first binding domain (recognizing e.g. CDH19, MSLN, DLL3, FLT3, EGFRvlll, BCMA, PSMA, CD33, CD19, CD70, CLDN18.2 or MUC17) and preferably also the structure and/or function of the second binding domain (recognizing CD3), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. Preferably, the first binding domain is characterized by the presence of three light chain CDRs (i.e. CDR1, CDR2 and CDR3 of the VL region) and/or three heavy chain CDRs (i.e. CDR1, CDR2 and CDR3 of the VH region). The second binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

In some embodiments, the first binding domain which binds to the target cell surface antigen and/or the second binding domain which binds to CD3ε is/are human binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

In some embodiments, the antigen binding protein comprises a single chain antibody construct. An scFv comprises a variable heavy chain, an scFv linker, and a variable light domain. Optionally, the C-terminus of the variable light chain is attached to the N-terminus of the scFv linker, the C-terminus of which is attached to the N-terminus of a variable heavy chain (N-vh-linker-vl-C), although the configuration can be switched (N-vl-linker-vh-C). Alternatively, the C-terminus of the variable heavy chain is attached to the N-terminus of the scFv linker, the C-terminus of which is attached to the N-terminus of a variable light chain (N-vl-linker-vh-C), although the configuration can be switched (N-vh-linker-v-C). Thus, specifically included in the depiction and description of scFvs are the scFvs in either orientation.

The at least two binding domains and the variable domains (VH/VL) of the antibody construct of the present disclosure may or may not comprise peptide linkers (spacer peptides). The term “peptide linker” comprises in accordance with the present invention an amino acid sequence by which the amino acid sequences of one (variable and/or binding) domain and another (variable and/or binding) domain of the antibody construct of the disclosure are linked with each other. The peptide linkers can also be used to fuse the third domain to the other domains of the antibody construct of the invention. An essential technical feature of such peptide linker is that it does not comprise any polymerization activity. Among the suitable peptide linkers are those described in U.S. Pat. Nos. 4,751,180 and 4,935,233 or WO 88/09344, the disclosure of which are incorporated herein by reference in their entireties. The peptide linkers can also be used to attach other domains or modules or regions (such as half-life extending domains) to the bispecific antibody construct described herein.

In some embodiments, the third domain comprises a “Fc” or “Fc region” or “Fc domain,” which refers to the polypeptide comprising the constant region of an antibody excluding the first constant region immunoglobulin domain. Thus, “Fc domain” refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). The bispecific antibody construct is preferably an IgG antibody (which includes several subclasses, including, but not limited to IgG1, IgG2, IgG3, and IgG4). Although the boundaries of the Fc region may vary, the human IgG heavy chain Fc region is usually defined to include residues C226 or P230 to its carboxyl-terminus, wherein the numbering is according to the EU index as in Kabat. In some embodiments, amino acid modifications are made to the Fc region, for example, to alter binding to one or more FcγR receptors or to the FcRn receptor.

In some embodiments, the formulations described herein comprise a bispecific antibody construct which binds human CD3 and human CDH19, or human CD3 and human MSLN, or human CD3 and human DLL3, or human CD3 and human FLT3, or human CD3 and human EGFRvIII, or human CD3 and human BCMA, or human CD3 and PSMA, or human CD3 and human CD33, or human CD3 and human CD19, human CD3 and human CD70, or human CD3 and human MUC17, or human CD3 and human CLDN18.2.

In some embodiments, the first binding domain of the bispecific antibody construct comprises a set of 6 CDRs set forth in (a) SEQ ID NOs: 24-29, (b) SEQ ID NOs: 34-39, (c) SEQ ID NOs: 78-83, (d) SEQ ID NOs: 10-15, (e) SEQ ID NOs: 46-51, (f) SEQ ID NOs: 88-93, (g) SEQ ID NOs: 67-72, (h) SEQ ID NOs: 56-61, (i) SEQ ID NOs: 112-117, (j) SEQ ID NOs: 100-105, (k) SEQ ID NOs:148-153, SEQ ID NOs: 157-162, or SEQ ID NOs: 166-171, or SEQ ID NOs: 175-180, (1) SEQ ID NOs:132-137, or (m) SEQ ID NOs: 123-128.

In some embodiments, the first binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 30, 40, 84, 16, 17, 52, 94, 73, 62, 118, 154, 163, 172, 181, 106, 138, 143, or 129. In some embodiments, the first binding domain of the bispecific antibody construct comprises a VH comprising the amino acid sequence set forth in SEQ ID NO: 30, 40, 84, 16, 17, 52, 94, 73, 62, 118, 154,163, 172, 181, 106, 138, 143, or 129.

In some embodiments, the first binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 31, 41, 85, 18, 19, 53, 95, 74, 63, 119, 155, 164, 173, 182, 107, 139, 144, or 130. In some embodiments, the first binding domain of the bispecific antibody construct comprises a VL comprising the amino acid sequence set forth in SEQ ID NO: 31, 41, 85, 18, 19, 53, 95, 74, 63, 119, 155, 164, 173, 182, 107, 139, 144, or 130.

In some embodiments, wherein the first binding domain comprises (a) a VH region comprising an amino acid sequence set forth in SEQ ID NO: 30 and a VL region comprising an amino acid sequence set forth in SEQ ID NO: 31; (b) a VH region comprising an amino acid sequence set forth in SEQ ID NO: 40 and a VL region comprising an amino acid sequence set forth in SEQ ID NO: 41; (c) a VH region comprising an amino acid sequence set forth in SEQ ID NO: 84 and a VL region comprising an amino acid sequence set forth in SEQ ID NO: 85; (d) a VH region comprising an amino acid sequence set forth in SEQ ID NO: 16 or 17 and a VL region comprising an amino acid sequence set forth in SEQ ID NO: 18 or 19; (e) a VH region comprising an amino acid sequence set forth in SEQ ID NO: 52 and a VL region comprising an amino acid sequence set forth in SEQ ID NO: 53; (f) a VH region comprising an amino acid sequence set forth in SEQ ID NO: 94 and a VL region comprising an amino acid sequence set forth in SEQ ID NO: 95; (g) a VH region comprising an amino acid sequence set forth in SEQ ID NO: 73 and a VL region comprising an amino acid sequence set forth in SEQ ID NO: 74; (h) a VH region comprising an amino acid sequence set forth in SEQ ID NO: 62 and a VL region comprising an amino acid sequence set forth in SEQ ID NO: 63; (i) a VH region comprising an amino acid sequence set forth in SEQ ID NO: 118 and a VL region comprising an amino acid sequence set forth in SEQ ID NO: 119; (j) a VH region comprising an amino acid sequence set forth in SEQ ID NO: 154, 163, 172, or 181 and a VL region comprising an amino acid sequence set forth in SEQ ID NO: 155, 164, 173 or 182; (k) a VH region comprising an amino acid sequence set forth in SEQ ID NO: 106 and a VL region comprising an amino acid sequence set forth in SEQ ID NO: 107; (1) a VH region comprising an amino acid sequence set forth in SEQ ID NO: 138 or 143, and a VL region comprising an amino acid sequence set forth in SEQ ID NO: 139 or 144; or (m) a VH region comprising an amino acid sequence set forth in SEQ ID NO: 129 and a VL region comprising an amino acid sequence set forth in SEQ ID NO: 130.

In some embodiments, the second binding domain of the bispecific antibody construct comprises a set of 6 CDRs set forth in SEQ ID NOs: 1-6.

In some embodiments, the second binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 7. In some embodiments, the second binding domain of the bispecific antibody construct comprises a VH comprising the amino acid sequence set forth in SEQ ID NO: 7.

In some embodiments, the second binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 8. In some embodiments, the second binding domain of the bispecific antibody construct comprises a VL comprising the amino acid sequence set forth in SEQ ID NO: 8.

In some embodiments, wherein the second binding domain comprises (a) a VH region comprising an amino acid sequence set forth in SEQ ID NO: 7 and a VL region comprising an amino acid sequence set forth in SEQ ID NO: 8.

In some embodiments, the bispecific antibody construct comprises a first binding domain that binds CD19 comprising an anti-CD19 variable light domain comprising the amino acid sequence of SEQ ID NO: 85 and an anti-CD19 variable heavy domain comprising the amino acid sequence of SEQ ID NO: 84, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 86 a second binding domain comprising the amino acid sequence of SEQ ID NO: 9. In some embodiments, the bispecific antibody construct comprises the amino acid sequence set forth in SEQ ID NO: 87.

In some embodiments, the bispecific antibody construct comprises a first binding domain that binds MSLN comprising an anti-MSLN variable light domain comprising the amino acid sequence of SEQ ID NO: 41 and an anti-MSLN variable heavy domain comprising the amino acid sequence of SEQ ID NO: 40, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 42, and a second binding domain comprising the amino acid sequence of SEQ ID NO: 9. In some embodiments, the bispecific antibody construct comprises an amino acid sequence set forth in SEQ ID NO: 43, 44 or 45.

In some embodiments, the bispecific antibody construct comprises a first binding domain that binds DLL3 comprising an anti-DLL3 variable light domain comprising the amino acid sequence of SEQ ID NO: 74 and an anti-DLL3 variable heavy domain comprising the amino acid sequence of SEQ ID NO: 73, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 75, and a second binding domain comprising the amino acid sequence of SEQ ID NO: 9. In some embodiments, the bispecific antibody construct comprises an amino acid sequence set forth in SEQ ID NO: 76 or 77.

In some embodiments, the bispecific antibody construct comprises a first binding domain that binds FLT3 comprising an anti-FLT3 variable light domain comprising the amino acid sequence of SEQ ID NO: 63 and an anti-FLT3 variable heavy domain comprising the amino acid sequence of SEQ ID NO: 62, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 64, a second binding domain comprising the amino acid sequence of SEQ ID NO: 9. In some embodiments, the bispecific antibody construct comprises an amino acid sequence set forth in SEQ ID NO: 65 or 66.

In some embodiments, the bispecific antibody construct comprises a first binding domain that binds EGFRvIII comprising an anti-EGFRvIII variable light domain comprising the amino acid sequence of SEQ ID NO: 31 and an anti-EGFRvIII variable heavy domain comprising the amino acid sequence of SEQ ID NO: 30, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 32, a second binding domain comprising the amino acid sequence of SEQ ID NO: 9. In some embodiments, the bispecific antibody construct comprises an amino acid sequence set forth in SEQ ID NO: 33.

In some embodiments, the bispecific antibody construct comprises a first binding domain that binds BCMA comprising an anti-BCMA variable light domain comprising the amino acid sequence of SEQ ID NO: 95 and an anti-BCMA variable heavy domain comprising the amino acid sequence of SEQ ID NO: 94, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 96, a second binding domain comprising the amino acid sequence of SEQ ID NO: 9. In some embodiments, the bispecific antibody construct comprises an amino acid sequence set forth in SEQ ID NO: 98 or SEQ ID NO: 97.

In some embodiments, the bispecific antibody construct comprises a first binding domain that binds PSMA comprising an anti-PSMA variable light domain comprising the amino acid sequence of SEQ ID NO: 119 or 107 and an anti-PSMA variable heavy domain comprising the amino acid sequence of SEQ ID NO: 118 or 106, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 120 or 108, a second binding domain comprising the amino acid sequence of SEQ ID NO: 9. In some embodiments, the bispecific antibody construct comprises an amino acid sequence set forth in SEQ ID NO: 121, 122, 109, 110 or 111.

In some embodiments, the bispecific antibody construct comprises a first binding domain that binds CD33 comprising an anti-CD33 variable light domain comprising the amino acid sequence of SEQ ID NO: 18 or 19 and an anti-CD33 variable heavy domain comprising the amino acid sequence of SEQ ID NO: 16 or 17, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 189 or 190, a second binding domain comprising the amino acid sequence of SEQ ID NO: 9. In some embodiments, the bispecific antibody construct comprises the amino acid sequence set forth in SEQ ID NO: 20, 21, 22 or 23.

In some embodiments, the bispecific antibody construct comprises a first binding domain that binds CDH19 comprising an anti-CDH19 variable light domain comprising the amino acid sequence of SEQ ID NO: 53 and an anti-CDH19 variable heavy domain comprising the amino acid sequence of SEQ ID NO: 52, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 54, a second binding domain comprising the amino acid sequence of SEQ ID NO: 9. In some embodiments, the bispecific antibody construct comprises the amino acid sequence set forth in SEQ ID NO: 55.

In some embodiments, the bispecific antibody construct comprises a first binding domain that binds MUC17 comprising an anti-MUC17 variable light domain comprising the amino acid sequence of SEQ ID NO: 155, 164, 173, or 182 and an anti-MUC17 variable heavy domain comprising the amino acid sequence of SEQ ID NO: 154, 163, 172, or 181 a second binding domain comprising an anti-CD3 variable heavy domain comprising the amino acid sequence of SEQ ID NO: 7, and an anti-CD3 variable light domain comprising the amino acid sequence of SEQ ID NO: 8. In some embodiments, the bispecific antibody construct comprises the amino acid sequence set forth in SEQ ID NO: 156, 165, 174 or 183.

In some embodiments, the bispecific antibody construct comprises a first binding domain that binds cldn18.2 comprising an anti-cldn18.2 variable light domain comprising the amino acid sequence of SEQ ID NO: 139 or 144 and an anti-cldn18.2 variable heavy domain comprising the amino acid sequence of SEQ ID NO: 138 or 143, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 140 or 145, and a second binding domain comprising the amino acid sequence of SEQ ID NO: 9. In some embodiments, the bispecific antibody construct comprises the amino acid sequence set forth in SEQ ID NO: 141, 142, 146 or 147.

In some embodiments, the bispecific antibody construct comprises a first binding domain that binds CD70 comprising an anti-CD70 variable light domain comprising the amino acid sequence of SEQ ID NO: 130 and an anti-CD70 variable heavy domain comprising the amino acid sequence of SEQ ID NO: 129, a second binding domain comprising an anti-CD3 variable heavy domain comprising the amino acid sequence of SEQ ID NO: 7, and an anti-CD3 variable light domain comprising the amino acid sequence of SEQ ID NO: 8. In some embodiments, the bispecific antibody construct comprises an amino acid sequence set forth in SEQ ID NO: 131.

In some embodiments, the formulations comprises an antigen-binding protein (e.g., bispecific antibody construct) described herein in an amount ranging from about 10 mg to about 50 mg (or from about 10 mg to about 20 mg, or about 20 mg to about 50 mg, or about 15 mg to about 20 mg, or about 20 mg to about 55 mg). In some embodiments, the formulation comprises a bispecific antibody construct in an amount of about 10 mg, about 15 mg, about 20 mg, about 25 mg, about 30 mg, about 35 mg, about 40 mg, about 45 mg, or about 50 mg.

In some embodiments, the formulation comprises an antigen-binding protein (e.g., bispecific antibody construct) in a concentration ranging from about 10 mg/mL to about 50 mg/mL (or from about 10 mg/mL to about 20 mg/mL or from about 15 mg/mL to about 20 mg/mL). In some embodiments, the formulation comprises a bispecific antibody construct n a concentration of about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL or about 50 mg/mL. In some embodiments, the formulation comprises a bispecific antibody construct in a concentration of about 20 mg/mL.

Buffers

The pharmaceutical formulation of the invention comprises a buffer, which optionally may be acetate, glutamate, citrate, succinate, tartrate, fumarate, maleate, histidine, phosphate, 2-(N-morpholino)ethanesulfonate or combinations thereof.

Buffering agents are often employed to control pH in the formulation. In some embodiments, the buffer is added in a concentration that maintains pH of the formulation of about 4 to about 6, about 4 to 5, or about 4.2. The effect of pH on formulations may be characterized using any one or more of several approaches such as accelerated stability studies and calorimetric screening studies (Remmele R. L. Jr., et al., Biochemistry, 38(16): 5241-7 (1999)).

The buffer system present in the formulation is selected to be physiologically compatible and to maintain a desired pH. The buffer may be present at a concentration between about 0.1 mM and about 1000 mM (1 M), or between about 5 mM and about 200 mM, or between about 5 mM to about 100 mM, or between about 10 mM and 50 about mM. Suitable buffer concentrations encompass concentrations of about 200 mM or less. In some embodiments, the buffer in the formulation is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM or about 5 mM. In some embodiments, the concentration of the buffer is at least 0.1, 0.5, 0.7, 0.8, 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some embodiments, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some embodiments, the concentration of the buffer is between 5, 6, 7, 8, 9, 10, 11, 12, 13, 14, 15, 16, 17, 18, 19, 20, 30, or 40 mM and 50 mM. In some embodiments, the concentration of the buffer is about 10 mM.

Surfactants

The pharmaceutical formulations described here comprise a surfactant. Exemplary surfactants include but are not limited to, polysorbate 20, polysorbate 40, polysorbate 60, polysorbate 80, poloxamer 188, poloxamer 407, triton X-100, polyoxyethylene, PEG 3350, PEG 4000, or a combination thereof.

Pharmaceutical formulations described herein comprise at least one surfactant, either individually or as a mixture in different ratios. In some embodiments, the formulation comprises a surfactant at a concentration of about 0.001% to about 5% w/v (or about 0.001% to about 0.5%, or about 0.004 to about 0.5% w/v or about 0.001 to about 0.01% w/v or about 0.004 to about 0.01% w/v). In some embodiments, the formulation comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some embodiments, the formulation comprises a surfactant at a concentration of about 0.001% to about 0.5% w/v. In some embodiments, the formulation comprises a surfactant at a concentration of about 0.001 to about 0.01% w/v. In some embodiments, the formulation comprises a surfactant at a concentration of about 0.001 to about 0.01% w/v. In some embodiments, the formulation comprises a surfactant at a concentration of about 0.001%, about 0.002%, about 0.003%, about 0.004%, about 0.005%, about 0.006%, about 0.007%, about 0.008%, about 0.009%, about 0.01%, about 0.05%, about 0.1%, about 0.2%, about 0.3%, about 0.4%, to about 0.5% w/v. In some embodiments, the formulation comprises a surfactant incorporated in a concentration of about 0.001% to about 0.01% w/v. In some embodiments, the surfactant is polysorbate 80 and the polysorbate 80 is present in a concentration of about 0.01% w/v.

Saccharides

The pharmaceutical formulations described herein comprise a saccharide. In some embodiments, the saccharide is a monosaccharide or a disaccharide. In some embodiments, the saccharide is glucose, galactose, fructose, xylose, sucrose, lactose, maltose, trehalose, sorbitol, mannitol or xylitol or a combination thereof.

In some embodiments, the pharmaceutical formulation comprises a saccharide at a concentration of about 0.01% to about 40% w/v, or about 00.1% to about 20% w/v, or about 1% to about 15%, or about 5% to about 12%, or about 7% to about 12% w/v. In some embodiments, the pharmaceutical formulation comprises at least one saccharide at a concentration of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, or at least 40% w/v. In some embodiments, the pharmaceutical formulation comprises at least one saccharide at a concentration of about 1%, about 2%, about 3%, about 4%, about 5%, about 6%, about 7%, about 8%, about 9%, about 10%, about 11%, about 12%, about 13%, about 14%, or about 15% w/v. In some embodiments, the pharmaceutical formulation comprises at least one saccharide at a concentration of about 1% to about 15% w/v. In a yet further embodiment, the pharmaceutical formulation comprises at least one saccharide at a concentration of about 7%, about 7.5%, about 8%, about 8.5%, about 9%, about 9.5%, about 10%, about 10.5%, about 11%, about 11.5%, or about 12% w/v. In some embodiments, the pharmaceutical formulation comprises at least one saccharide at a concentration of about 7% to about 12% w/v. In some embodiments, the at least one saccharide is in the formulation at a concentration of about 9% w/v. In some embodiments, the saccharide is sucrose and is present in the formulation ranging from about 9% to about 12% w/v.

In a preferred embodiment, the pharmaceutical formulation comprises 10 mM glutamate, 9% (w/V) sucrose and 0.01% (w/V) polysorbate 80, wherein the pH of the pharmaceutical formulation is 4.2. In some embodiments, the formulation is lyophilized.

Stability

The stability of a bispecific antibody construct formulation can be quantified in several ways. In some embodiments, stability of an antibody formulation is characterized by size exclusion high performance liquid chromatography (SE-HPLC), size exclusion ultra high performance liquid chromatography (SE-UHPLC), cation exchange high performance liquid chromatography (CE-HPLC), dynamic light scattering, analytical ultracentrifugation (AUC), field flow fractionation (FFF), isoelectric focusing and ion exchange chromatography (IEX). In some embodiments, stability of an antibody formulation is characterized by partial dissociation as measured by sodium-dodecyl sulfate capillary electrophoresis (CE-SDS) and/or sodium-dodecyl sulfate polyacrylamide gel electrophoresis (SDS-PAGE). In some embodiments, stability of the formulation is assessed by reduced capillary electrophoresis-sodium dodecyl sulfate (rCE-SDS). The rCE-SDS method separates the heavy chain (HC), light chain (LC), non-glycosylated HC (NGHC), and other minor peak species and groups under reducing conditions.

In some embodiments, stability of the formulation is characterized by the amount of high molecular weight (HMW) species of a bispecific antibody construct or by the rate of increase of the amount of HMW species of the bispecific antibody construct under storage conditions at various time points. In some embodiments, the amount of HMW species is determined at one week, two weeks, one months, three months, six months or twelve months in storage at approximately 4° C. or 40° C. In some embodiments, the rate of increase of HMW species is determined at one week, two weeks, one month, three months, six months or twelve months in storage at approximately 4° C. or 40° C. In some embodiments, the HMW species of a bispecific antibody construct in the formulation is measured by SE-UHPLC.

The stability of a bispecific antibody construct, and the capability of the formulation to maintain stability of the bispecific antibody construct, may be assessed over extended periods of time (e.g., weeks or months). In the context of a formulation, a stable formulation is one in which the bispecific antibody construct therein essentially retains its physical and/or chemical integrity and/or biological activity upon storage and during processes such as freeze/thaw, mechanical mixing and lyophilization. Bispecific antibody construct stability can be assessed, for example, by measuring the level and/or rate of formation of high molecular weight (HMW) aggregates, shift of charge profiles, and change in particle size.

In some embodiments, the relative values of any particular species of the bispecific antibody construct, as described herein, such as the intact BiTE® molecule or main species, or the high molecular weight (HMW) species (i.e., aggregates), or the low molecular weight (LMW) species (i.e., fragments), are expressed in relation to the respective values of the total product. For example, in some embodiments, the formulation is a lyophilized formulation, and 2.5% or less (e.g., 2.5%, or 2%, or 1.9%, or 1.8%, or 1.7%, or 1.6%, or 1.5%, or 1.4%, or 1.3%, or 1.2%, or 1.1%, or 1%, or 0.5%) of the bispecific antibody construct exists as HMW species in the lyophilized formulation. In some embodiments, the amount of HMW species in the lyophilized formulation increases less than 1%, (e.g., 0.9%, 0.8%, 0.7%, 0.6%, 0.5%, 0.4%, 0.3%, 0.2%, 0.1%) upon storage at 4° C. for one month or more (e.g., for one month, for three months, or for six months). In some embodiments, upon storage at 4° C. for one month or more (e.g., for one month, for three months, or for six months), the amount of HMW species in the formulation increases approximately between 0.1% and 0.4% (e.g., 0.1%, 0.2%, 0.3%, or 0.4%). In some embodiments, the amount of HMW species in the lyophilized formulation increases less than 1%, (e.g., 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, upon storage at 40° C. for one week or more (e.g., for one week, for two weeks, for one month or for three months) the amount of HMW species in the lyophilized formulation increases approximately between 0.1% and 0.7% (e.g., 0.1%, 0.2%, 0.3%, 0.4%, 0.5%, 0.6%, or 0.7%). In some embodiments, the HMW species of a bispecific antibody construct in the formulation is measured by SE-UHPLC.

In some embodiments, stability of the formulation is characterized by the amount of low molecular (LMW) species of a bispecific antibody construct or by the rate of increase of the amount of LMW species of the bispecific antibody construct under storage conditions at various time points. In some embodiments, the amount of LMW species is determined at one week, two weeks, one months, three months, six months or twelve months in storage at approximately 4° C. or 40° C. In some embodiments, the rate of increase of LMW species is determined at one week, two weeks, one month, three months, six months or twelve months in storage at approximately 4° C. or 40° C. In some embodiments, the LMW species of a bispecific antibody construct in the formulation is measured by reduced capillary electrophoresis-sodium dodecyl sulfate (rCE-SDS). In some embodiments, the LMW species of a bispecific antibody construct in the formulation is measured by Size Exclusion Chromatography (SEC).

In some embodiments, less than 2%, (e.g., 1.9%, 1.8%, 1.7%, 1.6%, 1.5%, 1.4%, 1.3%, 1.2%, 1.1%, 1%, or 0.5%) of the bispecific antibody construct exists as low molecular weight (LMW) species in the lyophilized formulation. In some embodiments, the amount of LMW species in the lyophilized formulation increases less than 2%, (e.g., 1.9%, 1.8%, 1.7%, 1.6%, 1.5%, 1.4%, 1.3%, 1.2%, 1.1%, 1%, or 0.5%) upon storage at 4° C. for one month or more (e.g., for one month, for three months, or for six months). In some embodiments, upon storage at 4° C. for one month or more (e.g., for one month, for three months, or for six months), the amount of LMW species in the formulation increases approximately between 0.1% and 0.7% (e.g., 0.1%, 0.2%, 0.3%, 0.4%, 0.5%, 0.6%, or 0.7%). In some embodiments, the amount of LMW species in the lyophilized formulation increases less than 1%, (e.g., 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, upon storage at 40° C. for one week or more (e.g., for one week, for two weeks, for one month or for three months) the amount of LMW species in the lyophilized formulation increases approximately between 0.1% and 0.7% (e.g., 0.1%, 0.2%, 0.3%, 0.4%, 0.5%, 0.6%, or 0.7%). In some embodiments, the LMW species of a bispecific antibody construct in the formulation is measured by Size Exclusion Chromatography (SEC). In some embodiments, the LMW species of a bispecific antibody construct in the formulation is measured by reduced capillary electrophoresis-sodium dodecyl sulfate (rCE-SDS).

In some embodiments, the percent of intact BiTE® molecule (i.e., main peak species) in the lyophilized formulation is greater than 95% of the total protein content in the formulation.

In some embodiments, the lyophilized formulation is stable upon storage at about 4° C. for one month, and the amount of HMW species in the formulation increases approximately between 0.1% to 0.7% (e.g., 0.1%, or 0.2%, or 0.3%, or 0.4%, or 0.5%, or 0.6%, or 0.7%), while in storage for at least one month. In some embodiments, the lyophilized formulation is stable upon storage at about 4° C. for three months, and the amount of HMW species in the formulation increases approximately between 0.0% to 0.2% (e.g., 0%, or 0.1%, or 0.2%), while in storage for at least three months. In some embodiments, the lyophilized formulation is stable upon storage at about 4° C. for six months, and the amount of HMW species in the formulation increases approximately between 0.0% to 0.4% (e.g., 0%, or 0.1%, or 0.2%, or 0.3%, or 0.4%), while in storage for at least six months. In some embodiments, the HMW species of a bispecific antibody construct in the formulation is measured by SE-UHPLC.

In one embodiment, the lyophilized formulation is stable upon storage at about 4° C. for one month, three months and six months, and the percent of intact BiTE® molecule is above 95% of the total protein content.

In some embodiments, the formulation is a liquid formulation and less than 3% (e.g., 2.5% or 2%, or 1.5%, or 1%, or 0.5%) of the bispecific antibody construct exists as HMW species in the liquid formulation. In some embodiments, the amount of HMW species in the liquid formulation increases less than 3% (e.g., 3%, 2.5%, 2%, 1%, or 0.5%) upon storage at 4° C. for one month or more (e.g., for one month, for three months, for six months, or for one year). In some embodiments, upon storage at 4° C. for one month or more (e.g., for one month, for three months, for six months or for one year), the amount of HMW species in the formulation increases approximately between 0.1% and 1% (e.g., 0.1%, or 0.2%, or 0.3%, or 0.4%, or 0.5%, or 0.6%, or 0.7%, or 0.8%, or 0.9%, or 1%). In some embodiments, the amount of HMW species in the liquid formulation increases less than 5% (e.g., 4.5%, or 4%, or 3.5%, or 3%, or 2.5%, or 2%, or 1.5%, or 1%, or 0.5% upon storage at 40° C. for one week or more (e.g., for one week, for two weeks, for one month or for three months). In some embodiments, upon storage at 40° C. for one week or more (e.g., for one week, for two weeks, for one month or for three months) the amount of HMW species in the liquid formulation increases approximately between 0.1% and 5% (e.g., 0.1%, or 0.2%, or 0.3%, or 0.4%, or 0.5%, or 0.6%, or 0.7%, or 0.8%, or 0.9%, or 1%, or 1.5%, or 2%, or 2.5%, or 3%, or 3.5%, or 4%, or 4.5% or 5%). In some embodiments, the HMW species of a bispecific antibody construct in the formulation is measured by SE-UHPLC.

In some embodiments, less than 2%, (e.g., 1.9%, or 1.8%, or 1.7%, or 1.6%, or 1.5%, or 1.4%, or 1.3%, or 1.2%, or 1.1%, or 1%, or 0.9%, or 0.8%, or 0.7%, or 0.6%, or 0.5%) of the bispecific antibody construct exists as low molecular weight (LMW) species in the liquid formulation. In some embodiments, the amount of LMW species in the liquid formulation increases less than 2%, (e.g., 1.9%, or 1.8%, or 1.7%, or 1.6%, or 1.5%, or 1.4%, or 1.3%, or 1.2%, or 1.1%, or 1%, or 0.5%, 0.4%, or 0.3%, or 0.2% or 0.1%) upon storage at 4° C. for one month or more (e.g., for one month, for three months, for six months, or for twelve months). In some embodiments, upon storage at 4° C. for one month or more (e.g., for one month, for three months, for six months, or for twelve months), the amount of LMW species in the liquid formulation increases approximately between 0.1% and 0.7% (e.g., 0.1%, 0.2%, 0.3%, 0.4%, 0.5%, 0.6%, or 0.7%). In some embodiments, the amount of LMW species in the liquid formulation increases less than 7%, (e.g., 6%, or 5%, or 4%, or 3%, or 2%, or 1%, or 0.9%, 0.8%, 0.7%, 0.6%, 0.5%, 0.4%, 0.3%, 0.2%, 0.1%) upon storage at 40° C. for one week or more (e.g., for one week, for two weeks, for one month or for three months). In some embodiments, upon storage at 40° C. for one week or more (e.g., for one week, for two weeks, for one month or for three months) the amount of LMW species in the lyophilized formulation increases approximately between 0.1% and 7% (e.g., 0.1%, 0.2%, 0.3%, 0.4%, 0.5%, 0.6%, or 0.7%, or 0.8%, or 0.9%, or 1%, or 1.5%, or 2%, or 3%, or 5%, or 6%, or 7%). In some embodiments, the LMW species of a bispecific antibody construct in the formulation is measured by Size Exclusion Chromatography (SEC). In some embodiments, the LMW species of a bispecific antibody construct in the formulation is measured by rCE-SDS.

In some embodiments, the percent of intact BiTE® molecule (i.e., main peak species) in the liquid formulation is greater than 96% of the total protein content in the formulation.

In some embodiments, the liquid formulation is stable upon storage at about 4° C. for one month, and the amount of HMW species in the formulation increases approximately between 0.1% to 0.4% (e.g., 0.1%, or 0.2%, or 0.3%, or 0.4%), while in storage for at least one month. In some embodiments, the liquid formulation is stable upon storage at about 4° C. for three months, and the amount of HMW species in the formulation increases approximately between 0.0% to 0.3% (e.g., 0%, or 0.1%, or 0.2%, or 0.3%), while in storage for at least three months. In some embodiments, the liquid formulation is stable upon storage at about 4° C. for six months, and the amount of HMW species in the formulation increases approximately between 0.0% to 0.6% (e.g., 0%, or 0.1%, or 0.2%, or 0.3%, or 0.4%, or 0.5%, or 0.6%), while in storage for at least six months. In some embodiments, the liquid formulation is stable upon storage at about 4° C. for twelve months, and the amount of HMW species in the formulation increases approximately between 0.0% to 0.2% (e.g., 0%, or 0.1%, or 0.2%), while in storage for at least twelve months. In some embodiments, the HMW species of a bispecific antibody construct in the formulation is measured by SE-UHPLC.

In one embodiment, the lyophilized formulation is stable upon storage at about 4° C. for one month, three months, six months, and twelve months, and the percent of intact BiTE® molecule is above 96% of the total protein content.

The stability of a formulation described herein can also be characterized by charge distribution, e.g., a change in the amount of the charge variant peaks of the antibody. For example, in some embodiments, the amount of acidic peak (e.g., deamidation, charge variants having a relatively lower isoelectric point (pI)) in the formulation increases by less than 2% (e.g., 2%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, or less) when stored at 4° C. for at least one month (e.g., for one month, three months, six months or twelve months). In some embodiments, the amount of basic peak (e.g., charge variants having a relatively higher pI) in the formulation increases by less than 6% (e.g., 6%, 5%, 4%, 3%, 2% or 1%) when stored at 4° C. for at least one month (e.g., for one month, three months, six months or twelve months). In some embodiments, the formulation is a lyophilized formulation and the amount of main peak in the formulation decreases by less than 4% (e.g., 4%, 3.5%, 3%, 2.5%, 2% 1% or less) when stored at 4° C. for at least one month. In some embodiments, the amount of main peak in the lyophilized formulation decreases by less than 6% (e.g., 6%, 5%, 4%, 3.5%, 3%, 2.5%, 2% or less) when stored at 4° C. for at least three months. In some embodiments, the amount of main peak in the lyophilized formulation decreases by less than 9% (e.g., 9%, 8%, 7%, 6%, 5%, 4%, 3.5%, 3%, 2.5%, 2% or less) when stored at 4° C. for at least six months. In some embodiments, the amount of main peak in the lyophilized formulation decreases by less than 9% (e.g., 9%, 8%, 7%, 6%, 5%, 4%, 3.5%, 3%, 2.5%, 2% or less) when stored at 4° C. for at least twelve months.

In some embodiments, the amount of acidic peak in the formulation increases by less than 30% (e.g., 30%, 25%, 20%, 15%, 10%, 9%, 8%, 7%, 6%, 4%, 4%, 3%, 2%, 1% or less) when stored at 40° C. for at least one week (e.g., for one week, two weeks, one month or three months). In some embodiments, the amount of basic peak (e.g., charge variants having a relatively higher pI) in the formulation increases by less than 15% (e.g., 15%, 10%, 9%, 8%, 7%, 6%, 4%, 4%, 3%, 2%, 1% or less), when stored at 40° C. for at least one week (e.g., for one week, two weeks, one month or three months). In some embodiments, the formulation is a lyophilized formulation and the amount of main peak in the formulation decreases by less than 4% (e.g., 4%, 3.5%, 3%, 2.5%, 2% 1% or less), when stored at 4° C. for at least one month. In some embodiments, the amount of main peak in the lyophilized formulation decreases by less than 6% (e.g., 6%, 5%, 4%, 3.5%, 3%, 2.5%, 2% or less), when stored at 4° C. for at least three months.

Therapeutic Use of the Formulation

The formulations described herein are useful as pharmaceutical formulations in the treatment, amelioration of cancer in a subject in need thereof. The terms “subject in need” or those “in need of treatment” includes those already with the disorder, as well as those in which the disorder is to be prevented. The “subject in need” or “patient” includes human and other mammalian subjects that receive either prophylactic or therapeutic treatment. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a tumor or cancer or a metastatic cancer as specified herein below, by the administration of formulation comprising an antigen-binding protein described herein to a subject in need thereof. Such an improvement may also be seen as a slowing or stopping of the progression of the tumor or cancer or metastatic cancer of the patient. The term “prevention” as used herein means the avoidance of the occurrence or re-occurrence of a patient having a tumor or cancer or a metastatic cancer as specified herein below, by the administration of the formulation comprising an antigen-binding protein (i.e., bispecific antibody construct) described herein to a subject in need thereof.

The disclosure provides a method of treating cancer, comprising administering to a subject in need thereof a therapeutically effective amount of a recombinant protein or a pharmaceutical formulation described herein. In certain embodiments, the subject is a human. In certain embodiments, the cancer is a solid tumor.

In some embodiments, the cancer is brain cancer, bladder cancer, breast cancer, clear cell kidney cancer, cervical cancer, colon and rectal cancer, endometrial cancer, gastric cancer, head/neck squamous cell carcinoma, lip and oral cancer, liver cancer, lung squamous cell carcinoma, melanoma, mesothelioma, non-small-cell lung cancer (NSCLC), non-melanoma skin cancer, ovarian cancer, oral cancer, pancreatic cancer, prostate cancer, renal cell carcinoma, sarcoma, small-cell lung cancer (SCLC), Squamous Cell Carcinoma of the Head and Neck (SCCHN), triple negative breast cancer, or thyroid cancer.

In some embodiments, the cancer is adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumor, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, or Wilms tumor.

In some embodiments, the cancer is acute lymphoblastic leukemia (ALL), acute myeloid leukemia (AML), chronic lymphocytic leukemia (CLL), or chronic myeloid leukemia (CML).

In some embodiments, the cancer is diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), or small lymphocytic lymphoma (SLL).

Indeed, cancers that can be treated include, but are not limited to, alveolar rhabdomyosarcoma, bone cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, esophageal cancer, gastrointestinal carcinoid tumor, hypopharynx cancer, larynx cancer, nasopharynx cancer, peritoneum, omentum, and mesentery cancer, pharynx cancer, small intestine cancer, soft tissue cancer, stomach cancer, testicular cancer, ureter cancer, and urinary bladder cancer.

Routes of Administration

Preferably, the pharmaceutical formulation is administered parenterally, e.g., intravenously, subcutaneously, or intramuscularly. Parenteral administration may be achieved by injection, such as bolus injection, or by infusion, such as continuous infusion. Administration may be achieved via depot for long-term release. In some embodiments, the formulation is administered intravenously by an initial bolus followed by a continuous infusion to maintain therapeutic circulating levels of drug product. In some embodiments, the formulation is administered as a one-time dose. Pharmaceutical formulations may be administered using a medical device. Examples of medical devices for administering pharmaceutical formulations are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

In some embodiments, the formulation is a lyophilized formulation, which is reconstituted either sterile water or suitable diluent for injection prior to administration.

The disclosure also contemplates uninterrupted administration of the suitable formulation. As a non-limiting example, uninterrupted or substantially uninterrupted, i.e., continuous administration may be realized by a small pump system worn by the patient for metering the influx of therapeutic agent into the body of the patient. The pharmaceutical formulation can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of the formulation may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable formulation into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical formulation to be administered can be between 0.1 and 50 ml.

Kits

As an additional aspect, the described herein are kits which comprise one or more pharmaceutical formulations described herein packaged in a manner which facilitates their use for administration to subjects. In one embodiment, such a kit includes a formulation described herein (e.g., a formulation comprising an antibody described therein), packaged in a container such as a sealed bottle, vessel, single-use or multi-use vial, prefilled syringe, or prefilled injection device, optionally with a label affixed to the container or included in the package that describes use of the compound or formulation in practicing the method. In one aspect, the formulation is packaged in a unit dosage form. The kit may further include a device suitable for administering the formulation according to a specific route of administration. Preferably, the kit contains a label that describes use of an antibody described herein or formulation described herein.

The pharmaceutical formulations described herein can be formulated in various forms, e.g., in solid, liquid, frozen, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts.

Generally, various storage and/or dosage forms are conceivable for the pharmaceutical formulation of the invention, depending, i.e., on the intended route of administration, delivery format and desired dosage (see, for example, Remington's Pharmaceutical Sciences, 22nd edition, Oslo, A., Ed., (2012)). The skilled person will be aware that such choice of a particular dosage form may for example influence the physical state, stability, rate of in vivo release and rate of in vivo clearance of an antibody.

For instance, the primary vehicle or carrier in a pharmaceutical formulation may be either aqueous or non-aqueous in nature. A suitable vehicle or carrier may be water for injection, physiological saline solution or artificial cerebrospinal fluid, possibly supplemented with other materials common in formulations for parenteral administration. Neutral buffered saline or saline mixed with serum albumin are further exemplary vehicles.

### EXAMPLES

### Example 1—Stability of the Liquid and Lyophilized Formulations Over Time

The stability of the following formulations was assessed by size exclusion high performance liquid chromatography (SE-HPLC) and cation exchange high performance liquid chromatography (CE-HPLC):

Liquid formulation: 20 mg/mL BITE® molecule, 10 mM L-glutamic acid, 9% (w/v) sucrose, 0.01% (w/v) Polysorbate 80, pH 4.2

Lyophilized formulation: 20 mg/mL BITE® molecule, 10 mM L-glutamic acid, 9% (w/v) sucrose, 0.01% (w/v) Polysorbate 80, pH 4.2

Table 1 below provides the percent high molecular weight (HMW) peak, main peak (monomers), and low molecular weight (LMW) species peak data for lyophilized and liquid formulations as assessed by SE-HPLC after storage at 4° C. for time 0, one month, three months, 6 months and 12 months.

TABLE 1
4° C.

| Sample | Timepoint | % HMW | % Main | % LMW |
|---|---|---|---|---|
| BiTE ®-I Liquid | 0 | 1.3 | 97.2 | 1.5 |
| | 1 M | 1.5 | 96.7 | 1.8 |
| | 3 M | 1.6 | 96.4 | 2.0 |
| | 6 M | 1.9 | 96.4 | 1.7 |
| | 12 M | 1.5 | 97.1 | 1.4 |
| BiTE ®-C Liquid | 0 | 1.7 | 97.0 | 1.4 |
| | 1 M | 1.2 | 97.1 | 1.7 |
| | 3 M | 1.6 | 96.4 | 2.0 |
| | 6 M | 1.6 | 96.3 | 2.1 |
| | 12 M | 1.4 | 96.7 | 1.9 |
| BiTE ®-G Liquid | 0 | 1.7 | 97.4 | 0.9 |
| | 1 M | 1.6 | 97.3 | 1.1 |
| | 3 M | 1.9 | 96.6 | 1.5 |
| | 6 M | 2.1 | 96.0 | 1.9 |
| | 12 M | 1.7 | 96.8 | 1.6 |
| BiTE ®-I Lyophilized | 0 | 1.8 | 96.5 | 1.6 |
| | 1 M | 2.5 | 95.9 | 1.6 |
| | 3 M | 1.8 | 96.3 | 1.9 |
| | 6 M | 1.8 | 96.9 | 1.4 |
| BiTE ®-C Lyophilized | 0 | 1.5 | 97.1 | 1.4 |
| | 1 M | 1.6 | 97.0 | 1.5 |
| | 3 M | 1.7 | 96.6 | 1.8 |
| | 6 M | 1.3 | 96.6 | 2.1 |
| BiTE ®-G Lyophilized | 0 | 1.8 | 97.3 | 0.9 |
| | 1 M | 1.9 | 97.1 | 1.0 |
| | 3 M | 1.9 | 96.8 | 1.2 |
| | 6 M | 2.2 | 96.4 | 1.5 |

Table 2 below provides the percent high molecular weight (HMW) peak, main (monomer) peak, and low molecular weight (LMW) species peak data for lyophilized and liquid formulations as assessed by SE-HPLC after storage at 40° C. for time 0, one week, two weeks, one month and three months.

40° C.

| Sample | Timepoint | % HMW | % Main | % LMW |
|---|---|---|---|---|
| BiTE ®-I Liquid | 1 Wk | 1.6 | 96.1 | 2.2 |
| | 2 Wk | 2.2 | 95.5 | 2.3 |
| | 1 M | 3.8 | 91.1 | 5.1 |
| | 3 M | 6.6 | 83.5 | 9.9 |
| BiTE ®-C Liquid | 1 Wk | 1.3 | 96.3 | 2.5 |
| | 2 Wk | 1.6 | 95.4 | 3.1 |
| | 1 M | 1.6 | 92.8 | 5.6 |
| | 3 M | 2.4 | 85.3 | 12.3 |
| BiTE ®-G Liquid | 1 Wk | 1.0 | 96.7 | 2.3 |
| | 2 Wk | 1.1 | 95.9 | 3.0 |
| | 1 M | 1.8 | 92.0 | 6.2 |
| | 3 M | 2.8 | 81.2 | 16.0 |
| BiTE ®-I Lyophilized | 1 Wk | 2.6 | 95.9 | 1.5 |
| | 2 Wk | 2.5 | 96.8 | 0.6 |
| | 1 M | 2.5 | 95.9 | 1.6 |
| | 3 M | 1.9 | 96.2 | 1.9 |
| BiTE ®-C Lyophilized | 1 Wk | 1.8 | 96.8 | 1.4 |
| | 2 Wk | 1.7 | 97.2 | 1.0 |
| | 1 M | 1.6 | 96.9 | 1.5 |
| | 3 M | 1.9 | 96.5 | 1.7 |
| BiTE ®-G Lyophilized | 1 Wk | 2.0 | 97.1 | 0.8 |
| | 2 Wk | 1.9 | 97.5 | 0.5 |
| | 1 M | 1.9 | 97.1 | 1.0 |
| | 3 M | 2.0 | 97.0 | 0.9 |

Cation exchange high performance liquid chromatography (CE-HPLC) was also performed for purity analysis to assess the charged variant distribution of the various BiTE® molecules in the tested formulation at 4° C. and 40° C. at various time. Table 3 below provides the percent main, acidic peak and basic peak data for the lyophilized and liquid formulations as assessed by CE-HPLC after storage at 4° C. for 0, one month, three months, six months and twelve months.

TABLE 3
4° C.

| Sample | Timepoint | % Acidic | % Main | % Basic |
|---|---|---|---|---|
| BiTE ®-I Liquid | 0 | 10.0 | 78.9 | 11.2 |
| | 1 M | 4.4 | 73.1 | 22.6 |
| | 3 M | 6.6 | 66.4 | 26.9 |
| | 6 M | 4.9 | 66.4 | 28.7 |
| | 12 M | 5.3 | 66.7 | 28.0 |
| BiTE ®-C Liquid | 0 | 3.6 | 92.8 | 3.6 |
| | 1 M | 4.2 | 88.7 | 7.1 |
| | 3 M | 5.2 | 87.4 | 7.4 |
| | 6 M | 4.1 | 86.1 | 9.8 |
| | 12 M | 4.9 | 85.9 | 9.2 |
| BiTE ®-G Liquid | 0 | 6.4 | 80.9 | 12.8 |
| | 1 M | 8.3 | 77.9 | 13.8 |
| | 3 M | 7.6 | 79.0 | 13.4 |
| | 6 M | 8.5 | 75.0 | 16.5 |
| | 12 M | 4.6 | 78.6 | 16.8 |
| BiTE ®-I Lyophilized | 0 | 9.9 | 78.4 | 11.7 |
| | 1 M | 4.2 | 78.4 | 17.4 |
| | 3 M | 6.3 | 67.4 | 26.3 |
| | 6 M | 4.4 | 69.0 | 26.6 |
| | 12 M | 4.4 | 70.6 | 25.0 |
| BiTE ®-C Lyophilized | 0 | 3.6 | 92.8 | 3.6 |
| | 1 M | 4.7 | 88.0 | 7.3 |
| | 3 M | 5.3 | 87.7 | 7.1 |
| | 6 M | 4.4 | 86.1 | 9.5 |
| | 12 M | 4.7 | 86.3 | 9.0 |
| BiTE ®-G Lyophilized | 0 | 6.4 | 80.5 | 13.1 |
| | 1 M | 8.3 | 77.0 | 14.7 |
| | 3 M | 7.5 | 81.0 | 11.5 |
| | 6 M | 6.5 | 84.0 | 9.5 |
| | 12 M | 5.1 | 80.3 | 14.6 |

Table 4 below provides the percent main peak, acidic peak and basic peak data for lyophilized and liquid formulations as assessed by CE-HPLC after storage at 40° C. for time 0, one week, two weeks, one month and three months.

TABLE 4
40° C.

| Sample | Timepoint | % Acidic | % Main | % Basic |
|---|---|---|---|---|
| BiTE ®-I Liquid | 0 | 0.0 | 100.0 | 0.0 |
| | 1 Wk | 12.4 | 75.3 | 12.4 |
| | 2 Wk | 10.9 | 74.5 | 14.6 |
| | 1 M | 8.5 | 69.5 | 22.0 |
| | 3 M | 28.1 | 34.2 | 37.7 |
| BiTE ®-C Liquid | 0 | 3.6 | 92.8 | 3.6 |
| | 1 Wk | 3.6 | 84.7 | 11.7 |
| | 2 Wk | 6.3 | 82.5 | 11.2 |
| | 1 M | 8.5 | 84.5 | 7.0 |
| | 3 M | 33.0 | 55.3 | 11.6 |
| BiTE ®-G Liquid | 0 | 6.4 | 80.9 | 12.8 |
| | 1 Wk | 6.7 | 78.9 | 14.4 |
| | 2 Wk | 9.0 | 68.5 | 22.5 |
| | 1 M | 20.7 | 50.0 | 29.2 |
| | 3 M | 40.5 | 24.6 | 34.9 |
| BiTE ®-I Lyophilized | 0 | 9.9 | 78.4 | 11.7 |
| | 1 Wk | 10.6 | 78.9 | 10.6 |
| | 2 Wk | 9.8 | 78.3 | 11.9 |
| | 1 M | 4.5 | 78.0 | 17.5 |
| | 3 M | 7.6 | 65.4 | 27.0 |
| BiTE ®-C Lyophilized | 0 | 3.6 | 92.8 | 3.6 |
| | 1 Wk | 33.0 | 55.4 | 11.6 |
| | 2 Wk | 3.8 | 86.3 | 9.9 |
| | 1 M | 4.8 | 88.1 | 7.1 |
| | 3 M | 6.3 | 86.0 | 7.6 |
| BiTE ®-G Lyophilized | 0 | 6.4 | 80.5 | 13.1 |
| | 1 Wk | 7.8 | 78.9 | 13.3 |
| | 2 Wk | 8.0 | 82.4 | 9.6 |
| | 1 M | 9.8 | 76.5 | 13.7 |
| | 3 M | 10.6 | 76.4 | 13.0 |

Reduced capillary electrophoresis-sodium dodecyl sulfate (rCE-SDS): The protein species are bound to SDS, an anionic detergent, and electokinetically injected into a bare fused silica capillary filled with SDS gel buffer. An electric voltage is applied across the capillary, under which the SDS coated proteins are separated by their difference in migration in a hydrophilic polymer-based solution. Proteins are detected by a photodiode array (PDA) detector as they pass through a UV detection window. Purity is evaluated by determining the percent corrected peak area of reach component. The rCE-SDS method separates the heavy chain (HC), light chain (LC), non-glycosylated HC (NGHC), and other minor peak species and groups under reducing conditions.

Size exclusion chromatography (SEC) separates molecules based on their size by filtration through a gel. The gel consists of spherical beads containing pores of a specific size distribution. Separation occurs when molecules of different sizes are included or excluded from the pores within the matrix. Small molecules diffuse into the pores and their flow through the column is retarded according to their size, while large molecules do not enter the pores and are eluted in the column's void volume. Consequently, molecules separate based on their size as they pass through the column and are eluted in order of decreasing molecular weight (MW). Operating conditions and gel selection depend on the application and the desired resolution.

Sub visible particle analysis is done via HIAC method. An electronic, liquid-borne particle-counting system containing a light-obscuration sensor with a liquid sampler quantifies the number of particles and their size range in a given test sample. When particles in a liquid pass, between the light source and the detector, they diminish or “obsure” the beam of light that falls on the detector. When the concentration of particle lies within the normal range of the sensor, these particles are detected one-by-one. The passage of each particle through the detection zone reduces the incident light on the photo-detector and the voltage output of the photo-detector is momentarily reduced. The changes in the voltage register as electrical pulses that are converted by the instrument into the numbers of particles present. The method is non-specific and measures particles regardless of their origin.

Finally, the moisture content of the lyophilized formulation was determined by a calorimetric titration with an oven. The Karl Fischer method's principle is based on the water content in the sample determined by means of calorimetric titration. Water is released by heating the sample in an oven. Dry air or inert gas such as nitrogen carried the evaporated moisture to the titrator. The amount of water present is determined by measuring the amount of coulombs (current/time) generated during the titration. When all the water has been consumed by titration, an excess of iodine occurs. The end point is indicted volumetrically by applying an alternating current of constant strength to a double Pt electrode. This results in a voltage difference between Pt wired of the indicator electrode, which is drastically lowered in the presence of minimal quantities of free iodine. This voltage difference is used to determine the end point of the titration. Moisture content of lyophilized formulations comprising BiTE®-I, BiTE®-C, and BiTE®-G were assessed after storage at 2-8° at time 0, one month, three months, six months and one year. As shown below in Table 5, none of the lyophilized formulations tested had greater than 1.70% water over all of the time periods tested.

TABLE 5

| BiTE ® Molecule | Time | % Water |
|---|---|---|
| BiTE ®-I | 0 | 0.7 |
| | 1 month | 1.1 |
| | 3 months | 1.6 |
| | 6 months | 0.1 |
| | 1 year | 1.1 |
| BiTE ®-C | 0 | 0.9 |
| | 1 month | 0.6 |
| | 3 months | 1.0 |
| | 6 months | 1.2 |
| | 1 year | 1.3 |
| BiTE ®-G | 0 | 0.8 |
| | 1 month | 1.0 |
| | 3 months | 1.1 |
| | 6 months | 1.2 |
| | 1 year | 1.5 |

### CONCLUSION

The present Example demonstrates that the high concentration formulations described herein are stable upon storage at about 4° C. for six months. In these stable formulations, the amount of HMW species increased approximately between 0.0% to 0.4%, while in storage for at least six months, without requiring the presence of a preservative or stabilizing agent.

What is claimed is:

1. A stable pharmaceutical formulation comprising a bispecific antibody construct in an amount of at least 10 mg/mL, a buffer, a saccharide, and a surfactant, wherein the formulation has a pH of 4-6.

2. The formulation of claim 1, that is lyophilized.

3. The formulation of claim 1 or 2, that is stable for up to 3 months at 4° C.

4. The formulation of any one of claims 1-3, wherein the formulation comprises less than 2% high molecular weight species after 3 months at 4° C.

5. The formulation of any one of claims 1-4, wherein the formulation has a pH of 4-5.

6. The formulation of any one of claims 1-4, wherein the formulation has a pH of 4.2.

7. The formulation of any one of claims 1-6, wherein the buffer is an acetate buffer, a glutamate buffer, a citrate buffer, a lactic buffer, a succinate buffer, a tartrate buffer, a fumarate buffer, a maleate buffer, a histidine buffer, or a phosphate buffer.

8. The formulation of any one of claims 1-7, wherein the buffer is present at a concentration ranging from 5 to 200 mM.

9. The formulation of claim 8, wherein the buffer is present at a concentration ranging from 10 to 50 mM.

10. The formulation of any one of claims 1-9, wherein the saccharide is a monosaccharide or a disaccharide.

11. The formulation of any one of claims 1-10, wherein the saccharide is glucose, galactose, fructose, xylose, sucrose, lactose, maltose, trehalose, sorbitol, mannitol or xylitol.

12. The formulation of any one of claims 1-11, wherein the saccharide is present in a concentration ranging from 1 to 15% (w/v).

13. The formulation of claim 12, wherein the saccharide is present in a concentration ranging from 5 to 12% (w/v).

14. The formulation of claim 12, wherein the saccharide is present in a concentration ranging from 7 to 12% (w/v).

15. The formulation of any one of claims 1-154 wherein the surfactant is polysorbate 20, polysorbate 40, polysorbate 60, polysorbate 80, poloxamer 188, poloxamer 407, triton X-100, polyoxyethylene, PEG 3350, PEG 4000, or a combination thereof.

16. The formulation of any one of claims 1-15, wherein the surfactant is present in a concentration ranging from 0.001% to 0.5% (w/v).

17. The formulation of claim 16, wherein the surfactant is present in a concentration ranging from 0.001% to 0.01% (w/v).

18. The formulation of any one of claims 1-17, wherein the bispecific antibody construct is present in a concentration ranging from 10 mg/mL to 20 mg/mL.

19. The formulation of any one of claims 1-18, wherein the bispecific antibody construct is present in an amount of 20 mg/mL.

20. The formulation of any one of claims 1-19, wherein the formulation comprises 10 mM glutamate, 9% (w/V) sucrose and 0.01% (w/V) polysorbate 80, wherein the formulation has a pH of 4.2.

21. The formulation of any one of claims 1-20, wherein the bispecific antibody construct comprises a first binding domain that binds to a target cell surface antigen, a second binding domain that binds to human CD3 on the surface of a T cell.

22. The formulation of claim 21, wherein the bispecific antibody construct further comprises a third domain comprising, in an amino to carboxyl order, hinge-CH2 domain-CH3 domain-linker-hinge-CH2 domain-CH3 domain.

22. The formulation of claim 21, wherein each of the first and second binding domains comprise a VH region and a VL region.

23. The formulation of claim 21 or claim 22, wherein the bispecific antibody construct is a single chain antibody construct.

24. The formulation of any one of claims 21-23, wherein the target cell surface antigen is CDH19, MSLN, DLL3, FLT3, EGFR, EGFRvlll, BCMA, PSMA, CD33, CD19, CD70, MUC17 or CLDN18.2.

25. The formulation of any one of claims 21-24, wherein the first binding domain of the bispecific antibody construct comprises a set of 6 CDRs set forth in (a) SEQ ID NOs: 24-29, (b) SEQ ID NOs: 34-39, (c) SEQ ID NOs: 78-83, (d) SEQ ID NOs: 10-15, (e) SEQ ID NOs: 46-51, (f) SEQ ID NOs: 88-93, (g) SEQ ID NOs: 67-72, (h) SEQ ID NOs: 56-61, (i) SEQ ID NOs: 112-117, (j) SEQ ID NOs: 100-105, (k) SEQ ID NOs:148-153, SEQ ID NOs: 157-162, SEQ ID NOs: 166-171, or SEQ ID NOs: 175-180, (1) SEQ ID NOs:132-137, or (m) 123-128.

26. The formulation of any one of claims 21-25, wherein the second binding domain of the bispecific antibody construct comprises a set of 6 CDRs set forth in SEQ ID NOs: 1-6.

27. The formulation of any one of claims 21-26, wherein the first binding domain comprises:
(a) a VH region comprising an amino acid sequence set forth in SEQ ID NO: 30 and a VL region comprising an amino acid sequence set forth in SEQ ID NO: 31;
(b) a VH region comprising an amino acid sequence set forth in SEQ ID NO: 40 and a VL region comprising an amino acid sequence set forth in SEQ ID NO: 41;
(c) a VH region comprising an amino acid sequence set forth in SEQ ID NO: 84 and a VL region comprising an amino acid sequence set forth in SEQ ID NO: 85;
(d) a VH region comprising an amino acid sequence set forth in SEQ ID NO: 16 or 17 and a VL region comprising an amino acid sequence set forth in SEQ ID NO: 18 or 19;
(e) a VH region comprising an amino acid sequence set forth in SEQ ID NO: 52 and a VL region comprising an amino acid sequence set forth in SEQ ID NO: 53;
(f) a VH region comprising an amino acid sequence set forth in SEQ ID NO: 94 and a VL region comprising an amino acid sequence set forth in SEQ ID NO: 95;
(g) a VH region comprising an amino acid sequence set forth in SEQ ID NO: 73 and a VL region comprising an amino acid sequence set forth in SEQ ID NO: 74;
(h) a VH region comprising an amino acid sequence set forth in SEQ ID NO: 62 and a VL region comprising an amino acid sequence set forth in SEQ ID NO: 63;
(i) a VH region comprising an amino acid sequence set forth in SEQ ID NO: 118 and a VL region comprising an amino acid sequence set forth in SEQ ID NO: 119;
(j) a VH region comprising an amino acid sequence set forth in SEQ ID NO: 154, 163, 172 or 181 and a VL region comprising an amino acid sequence set forth in SEQ ID NO: 155, 164, 173 or 182;
(k) a VH region comprising an amino acid sequence set forth in SEQ ID NO: 106 and a VL region comprising an amino acid sequence set forth in SEQ ID NO: 107;
(l) a VH region comprising an amino acid sequence set forth in SEQ ID NO: 138 or 143 and a VL region comprising an amino acid sequence set forth in SEQ ID NO: 139 or 144; or
(m) a VH region comprising an amino acid sequence set forth in SEQ ID NO: 129 and a VL region comprising an amino acid sequence set forth in SEQ ID NO: 130.

28. The formulation of any one of claims 21-27, wherein the second binding domain comprises a VH region comprising an amino acid sequence set forth in SEQ ID NO: 7 and a VL region comprising an amino acid sequence set forth in SEQ ID NO: 8.

29. The formulation of any one of claims 21-28, wherein the bispecific antibody construct comprises amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 55, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188.

30. A method of treating cancer in a subject in need thereof comprising administering to the subject the formulation of any one of claims 1-29.
